(19) United States
(12) Patent Application Publication
(10) Pub. No.: US 2026/0250697 A1
(43) Pub. Date: Aug. 27, 2026

(54) COMPOSITIONS AND METHODS FOR TARGETING DONOR POLYNUCELOTIDES IN SOYBEAN GENOMIC LOCI

(71) Applicant: PIONEER HI-BRED INTERNATIONAL, INC., JOHNSTON, IA (US)

(72) Inventors: SCOTT BETTS, DES MOINES, IA (US); HUIRONG GAO, JOHNSTON, IA (US); SIMON RENNY-BYFIELD, COLORADO SPRINGS, CO (US); JOHN BRYAN WOODWARD, AKENY, IA (US)

(73) Assignee: PIONEER HI-BRED INTERNATIONAL, INC., JOHNSTON, IA (US)

(21) Appl. No.: 18/861,682

(22) PCT Filed: May 23, 2023
(86) PCT No.: PCT/US23/67337

§ 371 Date: Oct. 30, 2024

Related U.S. Application Data
(60) Provisional application No. 63/346,112, filed on May 26, 2022.

(51) Int. Cl. C12N 15/82; C12N 9/22; C12N 15/11; C12Q 1/6895

(52) U.S. Cl. CPC C12N 15/8213; C12N 9/226; C12N 15/111; C12N 15/8242; C12Q 1/6895; C12N 2310/20; C12Q 2600/13

(57) ABSTRACT

Compositions and methods are provided for soybean genomic integration sites. These methods and compositions for producing a soybean plant comprise targeting the soybean genomic integration site with a site specific nuclease. The use of site specific nucleases for creating a double-strand-break target site can be performed with a zinc finger endonuclease, an engineered endonuclease, a meganuclease, a TALENs and/or a CRISPRCas endonuclease. The soybean genomic integration site can comprise at least one genomic locus of interest such as a trait cassette, a transgene, a mutated gene, a native gene, an edited gene or a site-specific integration (SSI) target site.

### INCORPORATION BY REFERENCE

The present application claims priority to International Patent Application No. PCT/US23/67337 filed on May 23, 2023, which claims priority to the benefit of U.S. Provisional Patent Application Ser. No. 63/346,112 filed on May 26, 2022, the disclosure of which is hereby incorporated by reference in its entirety.

### REFERENCE TO A SEQUENCE LISTING

The official copy of the sequence listing is submitted electronically via EFS-Web as an XML formatted sequence listing with a file named “9215-WO-PCT.xml”, created on May 11, 2023 with a size of 2.10 Mb, and is filed concurrently with the specification. The sequence listing contained in this XML formatted document is part of the specification and is herein incorporated by reference in its entirety.

### BACKGROUND

The genome of soybean plants were successfully transformed with transgenes in the early 1990's. Over the last thirty years, numerous methodologies have been developed for transforming the genome of soybean plants wherein a transgene is stably integrated into the genome of soybean plants. This evolution of transformation methodologies has resulted in the capability to successfully introduce a transgene comprising an agronomic trait within the genome of soybean plants. The introduction of insect resistance and herbicide tolerant traits within soybean plants in the late 1990's provided producers with a new and convenient technological innovation for controlling insects and a wide spectrum of weeds, which was unparalleled in cultivation farming methods. Currently, transgenic plants are commercially available throughout the world, and new transgenic products such as Enlist™ Soybean offer improved solutions for ever-increasing weed challenges. The utilization of transgenic soybean plants in modern agronomic practices would not be possible, but for the development and improvement of transformation methodologies.

However, current transformation methodologies rely upon the random insertion of transgenes within the genome of soybean plants. Reliance on random insertion of genes into a genome has several disadvantages. The transgenic events may randomly integrate within gene transcriptional sequences, thereby interrupting the expression of endogenous traits and altering the growth and development of the soybean plant. In addition, the transgenic events may indiscriminately integrate into locations of the genome that are susceptible to gene silencing, culminating in the reduced or complete inhibition of transgene expression either in the first or subsequent generations of transgenic soybean plants. Finally, the random integration of transgenes within the soybean plant genome requires considerable effort and cost in identifying the location of the transgenic event and selecting transgenic events that perform as designed without agronomic impact to the soybean plant. Novel assays must be continually developed to determine the precise location of the integrated transgene for each transgenic event. The random nature of plant transformation methodologies results in a “position-effect” of the integrated transgene, which hinders the effectiveness and efficiency of transformation methodologies.

Targeted genome modification of soybean plants has been a long-standing and elusive goal of both applied and basic research. Targeting genes and gene stacks to specific locations in the genome of soybean plants will improve the quality of transgenic events, reduce costs associated with production of transgenic events and provide new methods for making transgenic soybean plant products such as sequential gene stacking. Overall, targeting trangenes to specific genomic sites is likely to be commercially beneficial. Significant advances have been made in the last few years towards development of methods and compositions to target and cleave genomic DNA by site specific nucleases (e.g., Zinc Finger Nucleases (ZFNs), Meganucleases, Transcription Activator-Like Effector Nucelases (TALENS) and Clustered Regularly Interspaced Short Palindromic Repeats/CRISPR-associated nuclease (CRISPR Cas) with an engineered crRNA/tracr RNA), to induce targeted mutagenesis, induce targeted deletions of cellular DNA sequences, and facilitate targeted recombination of an exogenous donor DNA polynucleotide within a predetermined genomic locus. See, for example, U.S. Patent Publication No. 20030232410; 20050208489; 20050026157; 20050064474; and 20060188987, and International Patent Publication No. WO 2007/014275, the disclosures of which are incorporated by reference in their entireties for all purposes. U.S. Patent Publication No. 20080182332 describes use of non-canonical zinc finger nucleases (ZFNs) for targeted modification of plant genomes and U.S. Patent Publication No. 20090205083 describes ZFN-mediated targeted modification of a plant EPSPs genomic locus. Current methods for targeted insertion of exogenous DNA typically involve co-transformation of plant tissue with a donor DNA polynucleotide containing at least one transgene and a site specific nuclease (e.g., CRISPR) which is designed to bind and cleave a specific genomic locus of an actively transcribed coding sequence. This causes the donor DNA polynucleotide to stably insert within the cleaved genomic locus resulting in targeted gene addition at a specified genomic locus comprising an actively transcribed coding sequence.

An alternative approach is to target the transgene to preselected genomic integration site within the genome of soybean plants. In recent years, several technologies have been developed and applied to soybean plant cells for the targeted delivery of a transgene within the genome of soybean plants. However, much less is known about the attributes of genomic sites that are suitable for targeting. Historically, non-essential genes and pathogen (viral) integration sites in genomes have been used as loci for targeting. The number of such sites in genomes is rather limiting and there is therefore a need for identification and characterization of targetable optimal genomic loci that can be used for targeting of donor polynucleotide sequences. In addition to being amenable to targeting, genomic integration sites are expected to be neutral sites that can support transgene expression and soybean breeding applications. A need exists for compositions and methods that define criteria to identify genomic integration sites within the genome of soybean plants for targeted transgene integration.

### SUMMARY

Disclosed herein are sequences, constructs, and methods for a soybean genomic integration site. In certain aspects the integration site is greater than 5 Kb in size. In other aspects the integration site is low in genetic diversity, wherein the low genetic diversity comprises a haplotype with greater than 80% frequency. In further aspects the integration site is high in expected recombination frequencies, wherein the recombination rates are greater than 0.7 cM/1 Mb. In other aspects the integration site is in close proximity to a telomere, wherein the close proximity is less than 20 cM or less than 4.7 Mb from the end of a chromosome. In further aspects the genomic integration site comprises SEQ ID NO:206 or SEQ ID NO:207. In some aspects the genomic integration site comprises SEQ ID NO:1-109 or SEQ ID NO:264-334. In additional aspects the Genetic size of the low-diversity genomic region is less than 10 cM. In other aspects the Physical size of the low-diversity genomic region is less than 2.6 Mb. In an aspect the integration site comprises euchromatin. In further aspects the integration site is greater than 10 cM or 1.05 Mb in distance from heterochromatin. In additional aspects the range of the Haplotype frequency of the integration site is from 80 to 100%. In some aspects the integration site is greater than 5 cM from a QTL. In further aspects the integration site does not occur in a region that contains Structural Variation that inhibits genomic recombination. In other aspects the integration site comprises a gene expression cassette. For example, the gene expression cassette comprises an insecticidal resistance gene, herbicide tolerance gene, nitrogen use efficiency gene, water use efficiency gene, nutritional quality gene, DNA binding gene, and selectable marker gene. In other aspects the insertion site comprises at least one target site. Accordingly the target site is cleaved by a site specific nuclease. For example the nuclease is selected from the group consisting of a zinc finger nuclease, a CRISPR nuclease, a TALEN, a homing endonuclease or a meganuclease. In other aspects the insertion site sequence is modified during insertion of a donor DNA into said insertion site sequence.

Disclosed herein are soybean plants, soybean plant parts, or soybean plant cells comprising a recombinant sequence. In some aspects the soybean plants, soybean plant parts, or soybean plant cells comprise a soybean genomic integration site. In certain aspects the integration site of the soybean plant, soybean plant part or soybean plant cell is greater than 5 Kb in size. In other aspects the integration site of the soybean plant, soybean plant part or soybean plant cell is low in genetic diversity, wherein the low genetic diversity comprises a haplotype with greater than 80% frequency. In further aspects the integration site of the soybean plant, soybean plant part or soybean plant cell is high in expected recombination frequencies, wherein the recombination rates are greater than 0.7 cM/1 Mb. In other aspects the integration site of the soybean plant, soybean plant part or soybean plant cell is in close proximity to a telomere, wherein the close proximity is less than 20 cM or less than 4.7 Mb from the end of a chromosome. In further aspects the genomic integration site of the soybean plant, soybean plant part or soybean plant cell comprises SEQ ID NO:206 or SEQ ID NO:207. In some aspects the genomic integration site of the soybean plant, soybean plant part or soybean plant cell comprises SEQ ID NO: 1-109 or SEQ ID NO:264-334. In additional aspects the Genetic size of the low-diversity genomic region is less than 10 cM. In other aspects the Physical size of the low-diversity genomic region is less than 2.6 Mb. In an aspect the integration site of the soybean plant, soybean plant part or soybean plant cell comprises euchromatin. In further aspects the integration site of the soybean plant, soybean plant part or soybean plant cell is greater than 10 cM or 1.05 Mb in distance from heterochromatin. In additional aspects the range of the Haplotype frequency of the integration site of the soybean plant, soybean plant part or soybean plant cell is from 80 to 100%. In some aspects the integration site of the soybean plant, soybean plant part or soybean plant cell is greater than 5 cM from a QTL. In further aspects the integration site of the soybean plant, soybean plant part or soybean plant cell does not occur in a region that contains Structural Variation that inhibits genomic recombination. In other aspects the integration site of the soybean plant, soybean plant part or soybean plant cell comprises a gene expression cassette. For example, the gene expression cassette comprises an insecticidal resistance gene, herbicide tolerance gene, nitrogen use efficiency gene, water use efficiency gene, nutritional quality gene, DNA binding gene, and selectable marker gene. In other aspects the insertion site of the soybean plant, soybean plant part or soybean plant cell comprises at least one target site. Accordingly the target site is cleaved by a site specific nuclease. For example the nuclease is selected from the group consisting of a zinc finger nuclease, a CRISPR nuclease, a TALEN, a homing endonuclease or a meganuclease. In other aspects the insertion site sequence of the soybean plant, soybean plant part or soybean plant cell is modified during insertion of a donor DNA into said insertion site sequence of the soybean plant, soybean plant part or soybean plant cell.

Disclosed herein are sequences, constructs, and methods for making a transgenic soybean plant cell comprising a donor DNA targeted a genomic integration site. In some aspects the method for making a transgenic soybean plant cell comprises selecting a target site within the genomic integration site. In other aspects the method for making a transgenic soybean plant cell comprises introducing a site specific nuclease into a plant cell, wherein the site specific nuclease cleaves said target site. In further aspects the method for making a transgenic soybean plant cell comprises introducing the donor DNA into the plant cell. In some aspects the method for making a transgenic soybean plant cell comprises targeting the donor DNA into said target site of the genomic integration site, wherein the cleavage of said target site facilitates integration of the donor DNA into said target site. In other aspects the method for making a transgenic soybean plant cell comprises selecting transgenic plant cells comprising the donor DNA targeted to said target site of the genomic integration site. In further aspects the method for making a transgenic soybean plant cell comprises a donor DNA that comprises a gene expression cassette. For example, the gene expression cassette comprises an insecticidal resistance gene, herbicide tolerance gene, nitrogen use efficiency gene, water use efficiency gene, nutritional quality gene, DNA binding gene, and selectable marker gene. In further aspects the method for making a transgenic soybean plant cell comprises a site specific nuclease that is selected from the group consisting of a zinc finger nuclease, a CRISPR nuclease, a TALEN, a homing endonuclease or a meganuclease. In additional aspects the method for making a transgenic soybean plant cell comprises a donor DNA that is integrated within said target site via a homology directed repair integration method. In further aspects the method for making a transgenic soybean plant cell comprises a donor DNA that is integrated within said target site via a non-homologous end joining integration method. In some aspects the method for making a transgenic soybean plant cell comprises a genomic site that is greater than 1 cM in size. In other aspects the method for making a transgenic soybean plant cell comprises a genomic site that is low in genetic diversity, wherein the low genetic diversity comprises a haplotype with greater than 80% frequency. In further aspects the method for making a transgenic soybean plant cell comprises a genomic site that has high expected recombination frequencies, wherein the recombination rates are greater than 0.7 cM/1 Mb. In additional aspects the method for making a transgenic soybean plant cell comprises a genomic site that is close in proximity to a telomere, wherein the close proximity is less than 20 cM or less than 4.7 Mb from the end of a chromosome. In some aspects the method for making a transgenic soybean plant cell comprises a genomic site that of SEQ ID NO:206 or SEQ ID NO:207. In other aspects the method for making a transgenic soybean plant cell comprises a genomic site that of SEQ ID NO:1-109 or SEQ ID NO:264-334. In further aspects the method for making a transgenic soybean plant cell comprises a genomic site that a the low-diversity genomic region of less than 10 cM and ranges from 1 to 10 cM. In additional aspects the method for making a transgenic soybean plant cell comprises a genomic site with a Physical size of the low-diversity genomic region of less than 2.6 Mb and ranges from 785,899 bp to 954,789 bp. In some aspects the method for making a transgenic soybean plant cell comprises a genomic integration site comprises euchromatin. In other aspects the method for making a transgenic soybean plant cell comprises a genomic site is greater than 10 cM or 1.05 Mb in distance from heterochromatin. In further aspects the method for making a transgenic soybean plant cell comprises a genomic site, wherein the range of the Haplotype frequency is from 80 to 100%. In additional aspects the method for making a transgenic soybean plant cell comprises a genomic site, wherein the integration site is greater than 5 cM from a QTL. In some aspects the method for making a transgenic soybean plant cell comprises a genomic site that does not occur in a region that contains Structural Variation that inhibits genomic recombination. In further aspects the gene expression cassette expresses a gene. In some aspects the gene is expressed at a concentration of 1 to 100,000 ppm. In further aspects, the gene is expressed at 1, 2, 3, 4, 5, 6, 7, 8, 9, 10, 20, 30, 40, 50, 60, 70, 80, 90, 100, 200, 250, 300, 400, 500, 600, 700, 750, 800, 900, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 11000, 12000, 13000, 14000, 15000, 16000, 17000, 18000, 19000, 20000, 25000, 30000, 35000, 40000, 45000, 50000, 55000, 60000, 65000, 70000, 75000, 80000, 85000, 90000, 95000, or 10000 ppm. In other aspects the transgenic soybean plant cell is propagated into a transgenic soybean plant. The transgenic soybean plant is used to breed with another soybean plant to produce a progeny plant. In some aspects the breeding deploys the method of identifying a transgenic soybean plant cell comprising a donor DNA targeted to a genomic integration site; selecting soybean plants with the donor DNA at said genomic integration site; crossing said selected soybean plants to produce offspring; and, selecting offspring with desired traits associated at said genomic integration site.

Disclosed herein are sequences, constructs, and methods for identifying a soybean genomic integration site for site specific nuclease meditated integration of a donor polynucleotide. In some aspects of the method identifying a soybean genomic integration site for site specific nuclease meditated integration of a donor polynucleotide comprises obtaining a soybean genomic DNA sample from at least one soybean variety. In other aspects of the method identifying a soybean genomic integration site for site specific nuclease meditated integration of a donor polynucleotide comprises genotyping the soybean genomic DNA sample. In further aspects of the method identifying a soybean genomic integration site for site specific nuclease meditated integration of a donor polynucleotide comprises identifying haplotypes of the soybean genomic DNA sample. In other aspects of the method identifying a soybean genomic integration site for site specific nuclease meditated integration of a donor polynucleotide comprises assessing the genetic diversity of the haplotypes to identify haplotypes with low genetic diversity. In further aspects of the method identifying a soybean genomic integration site for site specific nuclease meditated integration of a donor polynucleotide comprises selecting haplotypes with low genetic diversity that comprises the following characteristics: 1. greater than 1 cM in size; 2. low genetic diversity, wherein the low genetic diversity comprises a haplotype with greater than 80% frequency; 3. high expected recombination frequencies, wherein the recombination frequencies are greater than 0.7 cM/1 Mb; and, 4. close proximity to a telomere, wherein the close proximity is less than 20 cM or less than 4.7 Mb from the end of a chromosome. In additional aspects of the method of identifying a soybean genomic integration site for site specific nuclease meditated integration of a donor polynucleotide comprises targeting the soybean genomic integration site with a site-specific nuclease to integrate a donor polynucleotide within the soybean genomic integration site. In other aspects of the method of identifying a soybean genomic integration site for site specific nuclease meditated integration of a donor polynucleotide comprises a gene expression cassette. In aspects of the method of identifying a soybean genomic integration site for site specific nuclease meditated integration the gene expression cassette comprises an insecticidal resistance gene, herbicide tolerance gene, nitrogen use efficiency gene, water use efficiency gene, nutritional quality gene, DNA binding gene, and/or selectable marker gene. In additional aspects of the method of identifying a soybean genomic integration site for site specific nuclease meditated integration the site specific nuclease is selected from the group consisting of a zinc finger nuclease, a CRISPR nuclease, a TALEN, a homing endonuclease or a meganuclease. In further aspects of the method of identifying a soybean genomic integration site for site specific nuclease meditated integration the donor DNA is integrated within said target site via a homology directed repair integration method. In some aspects of the method of identifying a soybean genomic integration site for site specific nuclease meditated integration the donor DNA is integrated within said target site via a non-homologous end joining integration method. In other aspects of the method of identifying a soybean genomic integration site for site specific nuclease meditated integration the genomic integration site comprises SEQ ID NO:206 or SEQ ID NO:207. In further aspects of the method of identifying a soybean genomic integration site for site specific nuclease meditated integration the genomic integration site comprises SEQ ID NO:1-109 or SEQ ID NO:264-334. In further aspects of the method of identifying a soybean genomic integration site for site specific nuclease meditated integration the Genetic size of the low-diversity genomic region is less than 10 cM and ranges from 1 to 10 cM. In additional aspects of the method of identifying a soybean genomic integration site for site specific nuclease meditated integration the Physical size of the low-diversity genomic region is less than 2.6 Mb and ranges from 785,899 bp to 954,789 bp. In some aspects of the method of identifying a soybean genomic integration site for site specific nuclease meditated integration the integration site comprises euchromatin. In other aspects of the method of identifying a soybean genomic integration site for site specific nuclease meditated integration the integration site is greater than 10 cM or 1.05 Mb in distance from heterochromatin. In further aspects of the method of identifying a soybean genomic integration site for site specific nuclease meditated integration the range of the Haplotype frequency is from 80 to 100%. In some aspects of the method of identifying a soybean genomic integration site for site specific nuclease meditated integration the integration site is greater than 5 cM from a QTL. In additional aspects of the method of identifying a soybean genomic integration site for site specific nuclease meditated integration the integration site does not occur in a region that contains Structural Variation that inhibits genomic recombination. In further aspects of the method of identifying a soybean genomic integration site for site specific nuclease meditated integration the genotyping of the soybean genomic DNA sample is completed by sequencing or analyzing SNP markers.

Disclosed herein are soybean plants, cells, plant parts and seeds comprising a transgene expression cassette that is inserted into a chromosomal locus in the soybean genome, wherein said chromosomal locus is located at SEQ ID NO:1-109, 206, 207 or 264-334 on chromosome 1 or chromosome 2, and wherein said transgene is expressed in said soybean plant.

### SEQUENCE LISTING

The nucleic acid sequences listed in the accompanying sequence listing are shown using standard letter abbreviations for nucleotide bases, as defined in 37 C.F.R. § 1.822. Only one strand of each nucleic acid sequence is shown, but the complementary strand and reverse complementary strand are understood as included by any reference to the displayed strand. As the complement and reverse complement of a primary nucleic acid sequence are necessarily disclosed by the primary sequence, the complementary sequence and reverse complementary sequence of a nucleic acid sequence are included by any reference to the nucleic acid sequence, unless it is explicitly stated to be otherwise (or it is clear to be otherwise from the context in which the sequence appears).

### DETAILED DESCRIPTION

### Definitions

Unless defined otherwise, all technical and scientific terms used herein have the same meaning as commonly understood by one of ordinary skill in the art to which this disclosure relates. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further clarify this disclosure, the following terms, abbreviations and definitions are provided.

As used herein, the terms “comprises”, “comprising”, “includes”, “including”, “has”, “having”, “contains”,” or “containing”, or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of an embodiment of the disclosure are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as disclosed in the application.

“The term “isolated”, as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “purified”, as used herein relates to the isolation of a molecule or compound in a form that is substantially free of contaminants normally associated with the molecule or compound in a native or natural environment, or substantially enriched in concentration relative to other compounds present when the compound is first formed, and means having been increased in purity as a result of being separated from other components of the original composition. The term “purified nucleic acid” is used herein to describe a nucleic acid sequence which has been separated, produced apart from, or purified away from other biological compounds including, but not limited to polypeptides, lipids and carbohydrates, while effecting a chemical or functional change in the component (e.g., a nucleic acid may be purified from a chromosome by removing protein contaminants and breaking chemical bonds connecting the nucleic acid to the remaining DNA in the chromosome).

The term “synthetic”, as used herein refers to a polynucleotide (i.e., a DNA or RNA) molecule that was created via chemical synthesis as an in vitro process. For example, a synthetic DNA may be created during a reaction within an Eppendorf™ tube, such that the synthetic DNA is enzymatically produced from a native strand of DNA or RNA. Other laboratory methods may be utilized to synthesize a polynucleotide sequence. Oligonucleotides may be chemically synthesized on an oligo synthesizer via solid-phase synthesis using phosphoramidites. The synthesized oligonucleotides may be annealed to one another as a complex, thereby producing a “synthetic” polynucleotide. Other methods for chemically synthesizing a polynucleotide are known in the art, and can be readily implemented for use in the present disclosure.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

For the purposes of the present disclosure, a “gene,” includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, introns and locus control regions.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein a “transgene” is defined to be a nucleic acid sequence that encodes a gene product, including for example, but not limited to, an mRNA. In one embodiment the transgene/heterologous coding sequence is an exogenous nucleic acid, where the transgene/heterologous coding sequence has been introduced into a host cell by genetic engineering (or the progeny thereof) where the transgene/heterologous coding sequence is not normally found. In one example, a transgene/heterologous coding sequence encodes an industrially or pharmaceutically useful compound, or a gene encoding a desirable agricultural trait (e.g., an herbicide-resistance gene). In yet another example, a transgene/heterologous coding sequence is an antisense nucleic acid sequence, wherein expression of the antisense nucleic acid sequence inhibits expression of a target nucleic acid sequence. In one embodiment the transgene/heterologous coding sequence is an endogenous nucleic acid, wherein additional genomic copies of the endogenous nucleic acid are desired, or a nucleic acid that is in the antisense orientation with respect to the sequence of a target nucleic acid in a host organism.

As used herein the term “non-GmPSID2 transgene” or “non-GmPSID2 gene” is any transgene/heterologous coding sequence that has less than 80% sequence identity with the GmPSID2 gene coding sequence.

As used herein, “heterologous DNA coding sequence” means any coding sequence other than the one that naturally encodes the GmPSID2 gene, or any homolog of the expressed GmPSID2 protein. The term “heterologous” is used in the context of this invention for any combination of nucleic acid sequences that is not normally found intimately associated in nature.

A “gene product” as defined herein is any product produced by the gene. For example the gene product can be the direct transcriptional product of a gene (e.g., mRNA, tRNA, rRNA, antisense RNA, interfering RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Gene expression can be influenced by external signals, for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein the term “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein, “homology-based gene silencing” (HBGS) is a generic term that includes both transcriptional gene silencing and post-transcriptional gene silencing. Silencing of a target locus by an unlinked silencing locus can result from transcription inhibition (transcriptional gene silencing; TGS) or mRNA degradation (post-transcriptional gene silencing; PTGS), owing to the production of double-stranded RNA (dsRNA) corresponding to promoter or transcribed sequences, respectively. The involvement of distinct cellular components in each process suggests that dsRNA-induced TGS and PTGS likely result from the diversification of an ancient common mechanism. However, a strict comparison of TGS and PTGS has been difficult to achieve because it generally relies on the analysis of distinct silencing loci. In some instances, a single transgene locus can triggers both TGS and PTGS, owing to the production of dsRNA corresponding to promoter and transcribed sequences of different target genes. Mourrain et al. (2007) Planta 225:365-79. It is likely that siRNAs are the actual molecules that trigger TGS and PTGS on homologous sequences: the siRNAs would in this model trigger silencing and methylation of homologous sequences in cis and in trans through the spreading of methylation of transgene sequences into the endogenous promoter.

As used herein, the term “nucleic acid molecule” (or “nucleic acid” or “polynucleotide”) may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide”. A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidites, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by the sequential addition of ribonucleotide-5′-triphosphates to the 3′ terminus of the growing chain (with a requisite elimination of the pyrophosphate). In either a linear or circular nucleic acid molecule, discrete elements (e.g., particular nucleotide sequences) may be referred to as being “upstream” or “5′” relative to a further element if they are bonded or would be bonded to the same nucleic acid in the 5′ direction from that element. Similarly, discrete elements may be “downstream” or “3” relative to a further element if they are or would be bonded to the same nucleic acid in the 3′ direction from that element.

A base “position”, as used herein, refers to the location of a given base or nucleotide residue within a designated nucleic acid. The designated nucleic acid may be defined by alignment (see below) with a reference nucleic acid.

Hybridization relates to the binding of two polynucleotide strands via Hydrogen bonds. Oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide and the DNA or RNA target. The oligonucleotide need not be 100% complementary to its target sequence to be specifically hybridizable. An oligonucleotide is specifically hybridizable when binding of the oligonucleotide to the target DNA or RNA molecule interferes with the normal function of the target DNA or RNA, and there is sufficient degree of complementarity to avoid non-specific binding of the oligonucleotide to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the chosen hybridization method and the composition and length of the hybridizing nucleic acid sequences. Generally, the temperature of hybridization and the ionic strength (especially the Na+ and/or Mg2+ concentration) of the hybridization buffer will contribute to the stringency of hybridization, though wash times also influence stringency. Calculations regarding hybridization conditions required for attaining particular degrees of stringency are discussed in Sambrook et al. (ed.), Molecular Cloning: A Laboratory Manual, 2nd ed., vol. 1-3, Cold Spring Harbor Laboratory Press, Cold Spring Harbor, New York, 1989, chs. 9 and 11.

As used herein, “stringent conditions” encompass conditions under which hybridization will only occur if there is less than 50% mismatch between the hybridization molecule and the DNA target. “Stringent conditions” include further particular levels of stringency. Thus, as used herein, “moderate stringency” conditions are those under which molecules with more than 50% sequence mismatch will not hybridize; conditions of “high stringency” are those under which sequences with more than 20% mismatch will not hybridize; and conditions of “very high stringency” are those under which sequences with more than 10% mismatch will not hybridize.

In particular embodiments, stringent conditions can include hybridization at 65° C., followed by washes at 65° C. with 0.1×SSC/0.1% SDS for 40 minutes.

The following are representative, non-limiting hybridization conditions:

Very High Stringency: Hybridization in 5×SSC buffer at 65° C. for 16 hours; wash twice in 2×SSC buffer at room temperature for 15 minutes each; and wash twice in 0.5×SSC buffer at 65° C. for 20 minutes each.

High Stringency: Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.

Moderate Stringency: Hybridization in 6×SSC buffer at room temperature to 55° C. for 16-20 hours; wash at least twice in 2×-3×SSC buffer at room temperature to 55° C. for 20-30 minutes each.

In particular embodiments, specifically hybridizable nucleic acid molecules can remain bound under very high stringency hybridization conditions. In these and further embodiments, specifically hybridizable nucleic acid molecules can remain bound under high stringency hybridization conditions. In these and further embodiments, specifically hybridizable nucleic acid molecules can remain bound under moderate stringency hybridization conditions.

As used herein, the term “oligonucleotide” refers to a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer”, which allows a DNA polymerase to extend the oligonucleotide and replicate the complementary strand.

The terms “percent sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

Global sequence alignment programs calculate the percent identity of two sequences by comparing alignments end-to-end in order to find exact matches, dividing the number of exact matches by the length of the shorter sequences, and then multiplying by 100. Basically, the percentage of identical nucleotides in a linear polynucleotide sequence of a reference (“query) polynucleotide molecule as compared to a test (“subject”) polynucleotide molecule when the two sequences are optimally aligned (with appropriate nucleotide insertions, deletions, or gaps).

Local sequence alignment programs are similar in their calculation, but only compare aligned fragments of the sequences rather than utilizing an end-to-end analysis. Local sequence alignment programs such as BLAST can be used to compare specific regions of two sequences. A BLAST comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST match is considered herein as having an E-value for the top BLAST hit of less than 1E-30; a medium BLASTX E-value is 1E-30 to 1E-8; and a low BLASTX E-value is greater than 1E-8. The protein function assignment in the present invention is determined using combinations of E-values, percent identity, query coverage and hit coverage. Query coverage refers to the percent of the query sequence that is represented in the BLAST alignment. Hit coverage refers to the percent of the database entry that is represented in the BLAST alignment. In one embodiment of the invention, function of a query polypeptide is inferred from function of a protein homolog where either (1) hit_p<1e-30 or % identity>35% AND query_coverage>50% AND hit_coverage>50%, or (2) hit_p<1e-8 AND query_coverage>70% AND hit_coverage>70%. The following abbreviations are produced during a BLAST analysis of a sequence.

| | |
|---|---|
| SEQ_NUM | provides the SEQ ID NO for the listed recombinant |
| | polynucleotide sequences. |
| CONTIG_ID | provides an arbitrary sequence name taken from the name of |
| | the clone from which the cDNA sequence was obtained. |
| PROTEIN_NUM | provides the SEQ ID NO for the recombinant polypeptide |
| | sequence |
| NCBI_GI | provides the GenBank ID number for the top BLAST hit for |
| | the sequence. The top BLAST hit is indicated by the National |
| | Center for Biotechnology Information GenBank Identifier |
| | number. |
| NCBI_GI_DESCRIPTION | refers to the description of the GenBank top BLAST hit for |
| | the sequence. |
| E_VALUE | provides the expectation value for the top BLAST match. |
| MATCH_LENGTH | provides the length of the sequence which is aligned in the |
| | top BLAST match |
| TOP_HIT_PCT_IDENT | refers to the percentage of identically matched nucleotides |
| | (or residues) that exist along the length of that portion of |
| | the sequences which is aligned in the top BLAST match. |
| CAT_TYPE | indicates the classification scheme used to classify the |
| | sequence. GO_BP = Gene Ontology Consortium -biological |
| | process; GO_CC = Gene Ontology Consortium - cellular |
| | component; GO_MF = Gene Ontology Consortium - molecular |
| | function; KEGG = KEGG functional hierarchy (KEGG = Kyoto |
| | Encyclopedia of Genes and Genomes); EC = Enzyme |
| | Classification from ENZYME data bank release 25.0; POI = |
| | Pathways of Interest. |
| CAT_DESC | provides the classification scheme subcategory to which the |
| | query sequence was assigned. |
| PRODUCT_CAT_DESC | provides the FunCAT annotation category to which the |
| | query sequence was assigned. |
| PRODUCT_HIT_DESC | provides the description of the BLAST hit which resulted in |
| | assignment of the sequence to the function category |
| | provided in the cat_desc column. |
| HIT_E | provides the E value for the BLAST hit in the hit_desc |
| | column. |
| PCT_IDENT | refers to the percentage of identically matched nucleotides |
| | (or residues) that exist along the length of that portion |
| | of the sequences which is aligned in the BLAST match |
| | provided in hit_desc. |
| QRY_RANGE | lists the range of the query sequence aligned with the hit. |
| HIT_RANGE | lists the range of the hit sequence aligned with the query. |
| QRY_CVRG | provides the percent of query sequence length that matches |
| | to the hit (NCBI) sequence in the BLAST match (% qry |
| | cvrg = (match length/query total length) × 100). |
| HIT_CVRG | provides the percent of hit sequence length that matches to |
| | the query sequence in the match generated using BLAST (% hit |
| | cvrg = (match length/hit total length) × 100). |

Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, CA). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (©1993-2016). DNASTAR. Madison, WI). The MegAlign program is global sequence alignment program for polynucleotides or proteins. In an embodiment, the subject disclosure relates to calculating percent identity between two polynucleotides or amino acid sequences using the Clustal suite of alignment programs, including, but not limited to, ClustalW and ClustalV (Higgins and Sharp (1988) Gene. Dec. 15; 73 (1): 237-44; Higgins and Sharp (1989) CABIOS 5:151-3; Higgins et al. (1992) Comput. Appl. Biosci. 8:189-91). In an embodiment, the subject disclosure relates to calculating percent identity between two polynucleotides or amino acid sequences using the GCG suite of programs (Wisconsin Package Version 9.0, Genetics Computer Group (GCG), Madison, WI). In an embodiment, the subject disclosure relates to calculating percent identity between two polynucleotides or amino acid sequences using the BLAST suite of alignment programs, for example, but not limited to, BLASTP, BLASTN, BLASTX, etc. (Altschul et al. (1990) J. Mol. Biol. 215:403-10). In an embodiment, the subject disclosure relates to calculating percent identity between two polynucleotides or amino acid sequences using the FASTA suite of alignment programs, including, but not limited to, FASTA, TFASTX, TFASTY, SSEARCH, LALIGN etc. (Pearson (1994) Comput. Methods Genome Res. [Proc. Int. Symp.], Meeting Date 1992 (Suhai and Sandor, Eds.), Plenum: New York, NY, pp. 111-20). In an embodiment, the subject disclosure relates to calculating percent identity between two polynucleotides or amino acid sequences using the T-Coffee alignment program (Notredame, et. al. (2000) J. Mol. Biol. 302, 205-17). In an embodiment, the subject disclosure relates to calculating percent identity between two polynucleotides or amino acid sequences using the DIALIGN suite of alignment programs, including, but not limited to DIALIGN, CHAOS, DIALIGN-TX, DIALIGN-T etc. (Al Ait, et. al. (2013) DIALIGN at GOBICS Nuc. Acids Research 41, W3-W7). In an embodiment, the subject disclosure relates to calculating percent identity between two polynucleotides or amino acid sequences using the MUSCLE suite of alignment programs (Edgar (2004) Nucleic Acids Res. 32 (5): 1792-1797). In an embodiment, the subject disclosure relates to calculating percent identity between two polynucleotides or amino acid sequences using the MAFFT alignment program (Katoh, et. al. (2002) Nucleic Acids Research 30 (14): 3059-3066). In an embodiment, the subject disclosure relates to calculating percent identity between two polynucleotides or amino acid sequences using the Genoogle program (Albrecht, Felipe. arXiv130702987v1 [cs.DC] 10 Jul. 2015). In an embodiment, the subject disclosure relates to calculating percent identity between two polynucleotides or amino acid sequences using the HMMER suite of programs (Eddy. (1998) Bioinformatics, 14:755-63). In an embodiment, the subject disclosure relates to calculating percent identity between two polynucleotides or amino acid sequences using the PLAST suite of alignment programs, including, but not limited to, TPLASTN, PLASTP, KLAST, and PLASTX (Nguyen & Lavenier. (2009) BMC Bioinformatics, 10:329). In an embodiment, the subject disclosure relates to calculating percent identity between two polynucleotides or amino acid sequences using the USEARCH alignment program (Edgar (2010) Bioinformatics 26 (19), 2460-61). In an embodiment, the subject disclosure relates to calculating percent identity between two polynucleotides or amino acid sequences using the SAM suite of alignment programs (Hughey & Krogh (Jan. 1995) Technical Report UCSCOCRL-95-7, University of California, Santa Cruz). In an embodiment, the subject disclosure relates to calculating percent identity between two polynucleotides or amino acid sequences using the IDF Searcher (O'Kane, K. C., The Effect of Inverse Document Frequency Weights on Indexed Sequence Retrieval, Online Journal of Bioinformatics, Volume 6 (2) 162-173, 2005). In an embodiment, the subject disclosure relates to calculating percent identity between two polynucleotides or amino acid sequences using the Parasail alignment program. (Daily, Jeff. Parasail: SIMD C library for global, semi-global, and local pairwise sequence alignments. BMC Bioinformatics. 17:18. Feb. 10, 2016). In an embodiment, the subject disclosure relates to calculating percent identity between two polynucleotides or amino acid sequences using the ScalaBLAST alignment program (Oehmen C, Nieplocha J. “ScalaBLAST: A scalable implementation of BLAST for high-performance data-intensive bioinformatics analysis.” IEEE Transactions on Parallel & Distributed Systems 17 (8): 740-749 Aug. 2006). In an embodiment, the subject disclosure relates to calculating percent identity between two polynucleotides or amino acid sequences using the SWIPE alignment program (Rognes, T. Faster Smilth-Waterman database searches with inter-sequence SIMD parallelization. BMC Bioiinformatics. 12, 221 (2011)). In an embodiment, the subject disclosure relates to calculating percent identity between two polynucleotides or amino acid sequences using the ACANA alignment program (Weichun Huang, David M. Umbach, and Leping Li, Accurate anchoring alignment of divergent sequences. Bioinformatics 22:29-34, Jan. 1 2006). In an embodiment, the subject disclosure relates to calculating percent identity between two polynucleotides or amino acid sequences using the DOTLET alignment program (Junier, T. & Pagni, M. DOTLET: diagonal plots in a web browser. Bioinformatics 16 (2): 178-9 Feb. 2000). In an embodiment, the subject disclosure relates to calculating percent identity between two polynucleotides or amino acid sequences using the G-PAS alignment program (Frohmberg, W., et al. G-PAS 2.0-an improved version of protein alignment tool with an efficient backtracking routine on multiple GPUs. Bulletin of the Polish Academy of Sciences Technical Sciences, Vol. 60, 491 Nov. 2012). In an embodiment, the subject disclosure relates to calculating percent identity between two polynucleotides or amino acid sequences using the GapMis alignment program (Flouri, T. et. al., Gap Mis: A tool for pairwise sequence alignment with a single gap. Recent Pat DNA Gene Seq. 7 (2): 84-95 Aug. 2013). In an embodiment, the subject disclosure relates to calculating percent identity between two polynucleotides or amino acid sequences using the EMBOSS suite of alignment programs, including, but not limited to: Matcher, Needle, Stretcher, Water, Wordmatch, etc. (Rice, P., Longden, I. & Bleasby, A. EMBOSS: The European Molecular Biology Open Software Suite. Trends in Genetics 16 (6) 276-77 (2000)). In an embodiment, the subject disclosure relates to calculating percent identity between two polynucleotides or amino acid sequences using the Ngila alignment program (Cartwright, R. Ngila: global pairwise alignments with logarithmic and affine gap costs. Bioinformatics. 23 (11): 1427-28. Jun. 1, 2007). In an embodiment, the subject disclosure relates to calculating percent identity between two polynucleotides or amino acid sequences using the probA, also known as propA, alignment program (Mückstein, U., Hofacker, IL, & Stadler, PF. Stochastic pairwise alignments. Bioinformatics 18 Suppl. 2: S153-60. 2002). In an embodiment, the subject disclosure relates to calculating percent identity between two polynucleotides or amino acid sequences using the SEQALN suite of alignment programs (Hardy, P. & Waterman, M. The Sequence Alignment Software Library at USC. 1997). In an embodiment, the subject disclosure relates to calculating percent identity between two polynucleotides or amino acid sequences using the SIM suite of alignment programs, including, but not limited to, GAP, NAP, LAP, etc. (Huang, X & Miller, W. A Time-Efficient, Linear-Space Local Similarity Algorithm. Advances in Applied Mathematics, vol. 12 (1991) 337-57). In an embodiment, the subject disclosure relates to calculating percent identity between two polynucleotides or amino acid sequences using the UGENE alignment program (Okonechnikov, K., Golosova, O. & Fursov, M. Unipro UGENE: a unified bioinformatics toolkit. Bioinformatics. 2012 28:1166-67). In an embodiment, the subject disclosure relates to calculating percent identity between two polynucleotides or amino acid sequences using the BAli-Phy alignment program (Suchard, MA & Redelings, BD. BAli-Phy: simultaneous Bayesian inference of alignment and phylogeny. Bioinformatics. 22:2047-48. 2006). In an embodiment, the subject disclosure relates to calculating percent identity between two polynucleotides or amino acid sequences using the Base-By-Base alignment program (Brodie, R., et. al. Base-By-Base: Single nucleotide-level analysis of whole viral genome alignments, BMC Bioinformatics, 5, 96, 2004). In an embodiment, the subject disclosure relates to calculating percent identity between two polynucleotides or amino acid sequences using the DECIPHER alignment program (ES Wright (2015) “DECIPHER: harnessing local sequence context to improve protein multiple sequence alignment.” BMC

Bioinformatics, doi: 10.1186/s12859-015-0749-z.). In an embodiment, the subject disclosure relates to calculating percent identity between two polynucleotides or amino acid sequences using the FSA alignment program (Bradley, R K, et. al. (2009) Fast Statistical Alignment. PLOS Computational Biology. 5: e1000392). In an embodiment, the subject disclosure relates to calculating percent identity between two polynucleotides or amino acid sequences using the Geneious alignment program (Kearse, M., et. al. (2012). Geneious Basic: an integrated and extendable desktop software platform for the organization and analysis of sequence data. Bioinformatics, 28 (12), 1647-49). In an embodiment, the subject disclosure relates to calculating percent identity between two polynucleotides or amino acid sequences using the Kalign alignment program (Lassmann, T. & Sonnhammer, E. Kalign—an accurate and fast multiple sequence alignment algorithm. BMC Bioinformatics 2005 6:298). In an embodiment, the subject disclosure relates to calculating percent identity between two polynucleotides or amino acid sequences using the MA VID alignment program (Bray, N. & Pachter, L. MAVID: Constrained Ancestral Alignment of Multiple Sequences. Genome Res. 2004 April; 14 (4): 693-99). In an embodiment, the subject disclosure relates to calculating percent identity between two polynucleotides or amino acid sequences using the MSA alignment program (Lipman, D J, et. al. A tool for multiple sequence alignment. Proc. Nat'l Acad. Sci. USA. 1989; 86:4412-15). In an embodiment, the subject disclosure relates to calculating percent identity between two polynucleotides or amino acid sequences using the MultAlin alignment program (Corpet, F., Multiple sequence alignment with hierarchial clustering. Nucl. Acids Res., 1988, 16 (22), 10881-90). In an embodiment, the subject disclosure relates to calculating percent identity between two polynucleotides or amino acid sequences using the LAGAN or MLAGAN alignment programs (Brudno, et. al. LAGAN and Multi-LAGAN: efficient tools for large-scale multiple alignment of genomic DNA. Genome Research 2003 April; 13 (4): 721-31). In an embodiment, the subject disclosure relates to calculating percent identity between two polynucleotides or amino acid sequences using the Opal alignment program (Wheeler, T. J., & Kececiouglu, J. D. Multiple alignment by aligning alignments. Proceedings of the 15th ISCB conference on Intelligent Systems for Molecular Biology. Bioinformatics. 23, i559-68, 2007). In an embodiment, the subject disclosure relates to calculating percent identity between two polynucleotides or amino acid sequences using the PicXAA suite of programs, including, but not limited to, PicXAA, PicXAA-R, PicXAA-Web, etc. (Mohammad, S., Sahraeian, E. & Yoon, B. PicXAA: greedy probabilistic construction of maximum expected accuracy alignment of multiple sequences. Nucleic Acids Research. 38 (15): 4917-28. 2010). In an embodiment, the subject disclosure relates to calculating percent identity between two polynucleotides or amino acid sequences using the PSAlign alignment program (SZE, S. H., Lu, Y., & Yang, Q. (2006) A polynomial time solvable formulation of multiple sequence alignment Journal of Computational Biology, 13, 309-19). In an embodiment, the subject disclosure relates to calculating percent identity between two polynucleotides or amino acid sequences using the StatAlign alignment program (Novák, A., et. al. (2008) StatAlign: an extendable software package for joint Bayesian estimation of alignments and evolutionary trees. Bioinformatics, 24 (20): 2403-04). In an embodiment, the subject disclosure relates to calculating percent identity between two polynucleotides or amino acid sequences using the Gap alignment program of Needleman and Wunsch (Needleman and Wunsch, Journal of Molecular Biology 48:443-453, 1970). In an embodiment, the subject disclosure relates to calculating percent identity between two polynucleotides or amino acid sequences using the BestFit alignment program of Smith and Waterman (Smith and Waterman, Advances in Applied Mathematics, 2:482-489, 1981, Smith et al., Nucleic Acids Research 11:2205-2220, 1983). These programs produces biologically meaningful multiple sequence alignments of divergent sequences. The calculated best match alignments for the selected sequences are lined up so that identities, similarities, and differences can be seen.

The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

The term “homology” is sometimes used to refer to the level of similarity between two or more nucleic acid or amino acid sequences in terms of percent of positional identity (i.e., sequence similarity or identity). Homology also refers to the concept of evolutionary relatedness, often evidenced by similar functional properties among different nucleic acids or proteins that share similar sequences.

As used herein, the term “variants” means substantially similar sequences. For nucleotide sequences, naturally occurring variants can be identified with the use of well-known molecular biology techniques, such as, for example, with polymerase chain reaction (PCR) and hybridization techniques as outlined herein.

For nucleotide sequences, a variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, variants of a particular nucleotide sequence of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular nucleotide sequence as determined by sequence alignment programs and parameters described elsewhere herein. A biologically active variant of a nucleotide sequence of the invention may differ from that sequence by as few as 1-15 nucleic acid residues, as few as 1-10, such as 6-10, as few as 5, as few as 4, 3, 2, or even 1 nucleic acid residue.

As used herein the term “operably linked” relates to a first nucleic acid sequence is operably linked with a second nucleic acid sequence when the first nucleic acid sequence is in a functional relationship with the second nucleic acid sequence. For instance, a promoter is operably linked with a coding sequence when the promoter affects the transcription or expression of the coding sequence. When recombinantly produced, operably linked nucleic acid sequences are generally contiguous and, where necessary to join two protein-coding regions, in the same reading frame. However, elements need not be contiguous to be operably linked.

As used herein, the term “promoter” refers to a region of DNA that generally is located upstream (towards the 5′ region of a gene) of a gene and is needed to initiate and drive transcription of the gene. A promoter may permit proper activation or repression of a gene that it controls. A promoter may contain specific sequences that are recognized by transcription factors. These factors may bind to a promoter DNA sequence, which results in the recruitment of RNA polymerase, an enzyme that synthesizes RNA from the coding region of the gene. The promoter generally refers to all gene regulatory elements located upstream of the gene, including, upstream promoters, 5′ UTR, introns, and leader sequences.

As used herein, the term “upstream-promoter” refers to a contiguous polynucleotide sequence that is sufficient to direct initiation of transcription. As used herein, an upstream-promoter encompasses the site of initiation of transcription with several sequence motifs, which include TATA Box, initiator sequence, TFIIB recognition elements and other promoter motifs (Jennifer, E. F. et al., (2002) Genes & Dev., 16:2583-2592). The upstream promoter provides the site of action to RNA polymerase II which is a multi-subunit enzyme with the basal or general transcription factors like, TFIIA, B, D, E, F and H. These factors assemble into a transcription pre initiation complex that catalyzes the synthesis of RNA from DNA template.

The activation of the upstream-promoter is done by the additional sequence of regulatory DNA sequence elements to which various proteins bind and subsequently interact with the transcription initiation complex to activate gene expression. These gene regulatory elements sequences interact with specific DNA-binding factors. These sequence motifs may sometimes be referred to as cis-elements. Such cis-elements, to which tissue-specific or development-specific transcription factors bind, individually or in combination, may determine the spatiotemporal expression pattern of a promoter at the transcriptional level. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, for example, Langridge et al., (1989) Proc. Natl. Acad. Sci. USA 86:3219-23. These cis-elements are located at a varying distance from transcription start point, some cis-elements (called proximal elements) are adjacent to a minimal core promoter region while other elements can be positioned several kilobases upstream or downstream of the promoter (enhancers).

As used herein, the terms “5′ untranslated region” or “5′ UTR” is defined as the untranslated segment in the 5′ terminus of pre-mRNAs or mature mRNAs. For example, on mature mRNAs, a 5′ UTR typically harbors on its 5′ end a 7-methylguanosine cap and is involved in many processes such as splicing, polyadenylation, mRNA export towards the cytoplasm, identification of the 5′ end of the mRNA by the translational machinery, and protection of the mRNAs against degradation.

As used herein, the term “intron” refers to any nucleic acid sequence comprised in a gene (or expressed polynucleotide sequence of interest) that is transcribed but not translated. Introns include untranslated nucleic acid sequence within an expressed sequence of DNA, as well as the corresponding sequence in RNA molecules transcribed therefrom. A construct described herein can also contain sequences that enhance translation and/or mRNA stability such as introns. An example of one such intron is the first intron of gene II of the histone H3 variant of Arabidopsis thaliana or any other commonly known intron sequence. Introns can be used in combination with a promoter sequence to enhance translation and/or mRNA stability.

As used herein, the terms “transcription terminator” or “terminator” is defined as the transcribed segment in the 3′ terminus of pre-mRNAs or mature mRNAs. For example, longer stretches of DNA beyond “polyadenylation signal” site is transcribed as a pre-mRNA. This DNA sequence usually contains transcription termination signal for the proper processing of the pre-mRNA into mature mRNA.

As used herein, the term “3′ untranslated region” or “3′ UTR” is defined as the untranslated segment in a 3′ terminus of the pre-mRNAs or mature mRNAs. For example, on mature mRNAs this region harbors the poly-(A) tail and is known to have many roles in mRNA stability, translation initiation, and mRNA export. In addition, the 3′ UTR is considered to include the polyadenylation signal and transcription terminator.

As used herein, the term “polyadenylation signal” designates a nucleic acid sequence present in mRNA transcripts that allows for transcripts, when in the presence of a poly-(A) polymerase, to be polyadenylated on the polyadenylation site, for example, located 10 to 30 bases downstream of the poly-(A) signal. Many polyadenylation signals are known in the art and are useful for the present invention. An exemplary sequence includes AAUAAA and variants thereof, as described in Loke J., et al., (2005) Plant Physiology 138 (3); 1457-1468.

A “DNA binding transgene” is a polynucleotide coding sequence that encodes a DNA binding protein. The DNA binding protein is subsequently able to bind to another molecule. A binding protein can bind to, for example, a DNA molecule (a DNA-binding protein), a RNA molecule (an RNA-binding protein), and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

Examples of DNA binding proteins include; meganucleases, zinc fingers, CRISPRs, and TALEN binding domains that can be “engineered” to bind to a predetermined nucleotide sequence. Typically, the engineered DNA binding proteins (e.g., zinc fingers, CRISPRs, or TALENs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP, CRISPR, and/or TALEN designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication Nos. 20110301073, 20110239315 and 20119145940.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; 6,534,261 and 6,794,136; see also WO 98/53058; WO 98/53059; WO 98/53060; WO 02/016536 and WO 03/016496.

In other examples, the DNA-binding domain of one or more of the nucleases comprises a naturally occurring or engineered (non-naturally occurring) TAL effector DNA binding domain. See, e.g., U.S. Patent Publication No. 20110301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than different effector proteins into the plant cell. Among these injected proteins are transcription activator-like (TALEN) effectors which mimic plant transcriptional activators and manipulate the plant transcriptome (see Kay et al., (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al., (1989) Mol Gen Genet 218:127-136 and WO2010079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al., (2006) J Plant Physiol 163 (3): 256-272). In addition, in the phytopathogenic bacteria Ralstonia solanacearum two genes, designated brg11 and hpx17 have been found that are homologous to the AvrBs3 family of Xanthomonas in the R. solanacearum biovar strain GMI1000 and in the biovar 4 strain RS1000 (See Heuer et al., (2007) Appl and Enviro Micro 73 (13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Patent Publication No. 20110301073, incorporated by reference in its entirety.

Specificity of these TAL effectors depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues at positions 12 and 13 with the identity of the contiguous nucleotides in the TAL-effector's target sequence (see Moscou and Bogdanove, (2009) Science 326:1501 and Boch et al., (2009) Science 326:1509-1512). Experimentally, the natural code for DNA recognition of these TAL-effectors has been determined such that an HD sequence at positions 12 and 13 leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al., ibid). Engineered TAL proteins have been linked to a FokI cleavage half domain to yield a TAL effector domain nuclease fusion (TALEN) exhibiting activity in a yeast reporter assay (plasmid based target).

The CRISPR (Clustered Regularly Interspaced Short Palindromic Repeats)/Cas (CRISPR Associated) nuclease system is a recently engineered nuclease system based on a bacterial system that can be used for genome engineering. It is based on part of the adaptive immune response of many bacteria and Archaea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 or Cas12fl nuclease to a region homologous to the crRNA in the target DNA called a “protospacer.” Cas9 or Cas12fl cleaves the DNA to generate blunt ends at the double-stranded break (DSB) at sites specified by a 20-nucleotide guide sequence contained within the crRNA transcript. Cas9 or Cas12fl requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 or Cas12fl nuclease to target any desired sequence (see Jinek et al., (2012) Science 337, pp. 816-821, Jinek et al., (2013), eLife 2: e00471, and David Segal, (2013) eLife 2: e00563). In other examples, the crRNA associates with the tracrRNA to guide the Cpf1 nuclease to a region homologous to the crRNA to cleave DNA with staggered ends (see Zetsche, Bernd, et al. Cell 163.3 (2015): 759-771.). Thus, the CRISPR/Cas system can be engineered to create a DSB at a desired target in a genome, and repair of the DSB can be influenced by the use of repair inhibitors to cause an increase in error prone repair.

In other examples, the DNA binding transgene/heterologous coding sequence is a site specific nuclease that comprises an engineered (non-naturally occurring) Meganuclease (also described as a homing endonuclease). The recognition sequences of homing endonucleases or meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al., (1997) Nucleic Acids Res. 25:3379-30 3388; Dujon et al., (1989) Gene 82:115-118; Perler et al., (1994) Nucleic Acids Res. 22, 11127; Jasin (1996) Trends Genet. 12:224-228; Gimble et al., (1996) J. Mol. Biol. 263:163-180; Argast et al., (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al., (2002) Molec. Cell 10:895-905; Epinat et al., (2003) Nucleic Acids Res. 5 31:2952-2962; Ashworth et al., (2006) Nature 441:656-659; Paques et al., (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

As used herein, the term “transformation” encompasses all techniques that a nucleic acid molecule can be introduced into such a cell. Examples include, but are not limited to: transfection with viral vectors; transformation with plasmid vectors; electroporation; lipofection; microinjection (Mueller et al., (1978) Cell 15:579-85); Agrobacterium-mediated transfer; direct DNA uptake; WHISKERS™-mediated transformation; and microprojectile bombardment. These techniques may be used for both stable transformation and transient transformation of a plant cell. “Stable transformation” refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” refers to the introduction of a nucleic acid fragment into the nucleus, or DNA-containing organelle, of a host organism resulting in gene expression without genetically stable inheritance.

An exogenous nucleic acid sequence. In one example, a transgene/heterologous coding sequence is a gene sequence (e.g., an herbicide-resistance gene), a gene encoding an industrially or pharmaceutically useful compound, or a gene encoding a desirable agricultural trait. In yet another example, the transgene/heterologous coding sequence is an antisense nucleic acid sequence, wherein expression of the antisense nucleic acid sequence inhibits expression of a target nucleic acid sequence. A transgene/heterologous coding sequence may contain regulatory sequences operably linked to the transgene/heterologous coding sequence (e.g., a promoter). In some embodiments, a polynucleotide sequence of interest is a transgene. However, in other embodiments, a polynucleotide sequence of interest is an endogenous nucleic acid sequence, wherein additional genomic copies of the endogenous nucleic acid sequence are desired, or a nucleic acid sequence that is in the antisense orientation with respect to the sequence of a target nucleic acid molecule in the host organism.

As used herein, the term a transgenic “event” is produced by transformation of plant cells with heterologous DNA, i.e., a nucleic acid construct that includes a transgene/heterologous coding sequence of interest, regeneration of a population of plants resulting from the insertion of the transgene/heterologous coding sequence into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene/heterologous coding sequence DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene/heterologous coding sequence of interest as the result of a sexual cross of one parental line that includes the inserted DNA (e.g., the original transformant and progeny resulting from selfing) and a parental line that does not contain the inserted DNA.

As used herein, the terms “Polymerase Chain Reaction” or “PCR” define a procedure or technique in which minute amounts of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51:263 (1987); Erlich, ed., PCR Technology, (Stockton Press, NY, 1989).

As used herein, the term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

As used herein, the term “probe” refers to an oligonucleotide that hybridizes to a target sequence. In the TaqMan® or TaqMan®-style assay procedure, the probe hybridizes to a portion of the target situated between the annealing site of the two primers. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides. A probe can further include a detectable label, e.g., a fluorophore (Texas-Red®, Fluorescein isothiocyanate, etc.,). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc.

As used herein, the terms “restriction endonucleases” and “restriction enzymes” refer to bacterial enzymes, each of which cut double-stranded DNA at or near a specific nucleotide sequence. Type-2 restriction enzymes recognize and cleave DNA at the same site, and include but are not limited to XbaI, BamHI, HindIII, EcoRI, XhoI, SalI, KpnI, Aval, PstI and Smal.

As used herein, the term “vector” is used interchangeably with the terms “construct”, “cloning vector” and “expression vector” and means the vehicle by which a DNA or RNA sequence (e.g. a foreign gene) can be introduced into a host cell, so as to transform the host and promote expression (e.g. transcription and translation) of the introduced sequence. A “non-viral vector” is intended to mean any vector that does not comprise a virus or retrovirus. In some embodiments a “vector” is a sequence of DNA comprising at least one origin of DNA replication and at least one selectable marker gene. Examples include, but are not limited to, a plasmid, cosmid, bacteriophage, bacterial artificial chromosome (BAC), or virus that carries exogenous DNA into a cell. A vector can also include one or more genes, antisense molecules, and/or selectable marker genes and other genetic elements known in the art. A vector may transduce, transform, or infect a cell, thereby causing the cell to express the nucleic acid molecules and/or proteins encoded by the vector.

The term “plasmid” defines a circular strand of nucleic acid capable of autosomal replication in either a prokaryotic or a eukaryotic host cell. The term includes nucleic acid which may be either DNA or RNA and may be single- or double-stranded. The plasmid of the definition may also include the sequences which correspond to a bacterial origin of replication.

As used herein, the term “selectable marker gene” as used herein defines a gene or other expression cassette which encodes a protein which facilitates identification of cells into which the selectable marker gene is inserted. For example a “selectable marker gene” encompasses reporter genes as well as genes used in plant transformation to, for example, protect plant cells from a selective agent or provide resistance/tolerance to a selective agent. In one embodiment only those cells or plants that receive a functional selectable marker are capable of dividing or growing under conditions having a selective agent. The phrase “marker-positive” refers to plants that have been transformed to include a selectable marker gene.

As used herein, the term “detectable marker” refers to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In an embodiment, a detectable marker can be attached by spacer arms of various lengths to reduce potential steric hindrance.

As used herein, the terms “cassette”, “expression cassette” and “gene expression cassette” refer to a segment of DNA that can be inserted into a nucleic acid or polynucleotide at specific restriction sites or by homologous recombination. As used herein the segment of DNA comprises a polynucleotide that encodes a polypeptide of interest, and the cassette and restriction sites are designed to ensure insertion of the cassette in the proper reading frame for transcription and translation. In an embodiment, an expression cassette can include a polynucleotide that encodes a polypeptide of interest and having elements in addition to the polynucleotide that facilitate transformation of a particular host cell. In an embodiment, a gene expression cassette may also include elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

As used herein a “linker” or “spacer” is a bond, molecule or group of molecules that binds two separate entities to one another. Linkers and spacers may provide for optimal spacing of the two entities or may further supply a labile linkage that allows the two entities to be separated from each other. Labile linkages include photocleavable groups, acid-labile moieties, base-labile moieties and enzyme-cleavable groups. The terms “polylinker” or “multiple cloning site” as used herein defines a cluster of three or more Type-2 restriction enzyme sites located within 10 nucleotides of one another on a nucleic acid sequence. In other instances the term “polylinker” as used herein refers to a stretch of nucleotides that are targeted for joining two sequences via any known seamless cloning method (i.e., Gibson Assembly®, NEBuilder HiFID NA Assembly®, Golden Gate Assembly, BioBrick® Assembly, etc.). Constructs comprising a polylinker are utilized for the insertion and/or excision of nucleic acid sequences such as the coding region of a gene.

A “centimorgan” (cM) or “map unit” is the distance between two linked genes, markers, target sites, genomic loci of interest, loci, or any pair thereof, wherein 1% of the products of meiosis are recombinant. Thus, a centimorgan is equivalent to a distance equal to a 1% average recombination frequency between the two linked genes, markers, target sites, loci, genomic loci of interest or any pair thereof.

As used herein, the term “control” refers to a sample used in an analytical procedure for comparison purposes. A control can be “positive” or “negative”. For example, where the purpose of an analytical procedure is to detect a differentially expressed transcript or polypeptide in cells or tissue, it is generally preferable to include a positive control, such as a sample from a known plant exhibiting the desired expression, and a negative control, such as a sample from a known plant lacking the desired expression.

As used herein, the term “plant” includes a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein.

As used herein, the term “small RNA” refers to several classes of non-coding ribonucleic acid (ncRNA). The term small RNA describes the short chains of ncRNA produced in bacterial cells, animals, plants, and fungi. These short chains of ncRNA may be produced naturally within the cell or may be produced by the introduction of an exogenous sequence that expresses the short chain or ncRNA. The small RNA sequences do not directly code for a protein, and differ in function from other RNA in that small RNA sequences are only transcribed and not translated. The small RNA sequences are involved in other cellular functions, including gene expression and modification. Small RNA molecules are usually made up of about 20 to 30 nucleotides. The small RNA sequences may be derived from longer precursors. The precursors form structures that fold back on each other in self-complementary regions; they are then processed by the nuclease Dicer in animals or DCL1 in plants.

Many types of small RNA exist either naturally or produced artificially, including microRNAs (miRNAs), short interfering RNAs (siRNAs), antisense RNA, short hairpin RNA (shRNA), and small nucleolar RNAs (snoRNAs). Certain types of small RNA, such as microRNA and siRNA, are important in gene silencing and RNA interference (RNAi). Gene silencing is a process of genetic regulation in which a gene that would normally be expressed is “turned off” by an intracellular element, in this case, the small RNA. The protein that would normally be formed by this genetic information is not formed due to interference, and the information coded in the gene is blocked from expression.

As used herein, the term “small RNA” encompasses RNA molecules described in the literature as “tiny RNA” (Storz, (2002) Science 296:1260-3; Illangasekare et al., (1999) RNA 5:1482-1489); prokaryotic “small RNA” (sRNA) (Wassarman et al., (1999) Trends Microbiol. 7:37-45); eukaryotic “noncoding RNA (ncRNA)”; “micro-RNA (miRNA)”; “small non-mRNA (snmRNA)”; “functional RNA (fRNA)”; “transfer RNA (tRNA)”; “catalytic RNA” [e.g., ribozymes, including self-acylating ribozymes (Illangaskare et al., (1999) RNA 5:1482-1489); “small nucleolar RNAs (snoRNAs),” “tmRNA” (a.k.a. “10S RNA,” Muto et al., (1998) Trends Biochem Sci. 23:25-29; and Gillet et al., (2001) Mol Microbiol. 42:879-885); RNAi molecules including without limitation “small interfering RNA (siRNA),” “endoribonuclease-prepared siRNA (e-siRNA),” “short hairpin RNA (shRNA),” and “small temporally regulated RNA (stRNA),” “diced siRNA (d-siRNA),” and aptamers, oligonucleotides and other synthetic nucleic acids that comprise at least one uracil base.

Unless otherwise specifically explained, all technical and scientific terms used herein have the same meaning as commonly understood by those of ordinary skill in the art to which this disclosure belongs. Definitions of common terms in molecular biology can be found in, for example: Lewin, Genes V, Oxford University Press, 1994 (ISBN 0-19-854287-9); Kendrew et al. (eds.), The Encyclopedia of Molecular Biology, Blackwell Science Ltd., 1994 (ISBN 0-632-02182-9); and Meyers (ed.), Molecular Biology and Biotechnology: A Comprehensive Desk Reference, VCH Publishers, Inc., 1995 (ISBN 1-56081-569-8).

### Embodiments

In some embodiment the subject disclosure relates to soybean plant genomic integration sites. Aspects of the disclosure include both compositions and methods.

In an embodiment the soybean plant genomic integration site comprises a polynucleotide that is at least 1 centimorgans (CM) in length. In aspects of this embodiment the genomic integration site may be 1 cM, 2 cM, 3 cM, 4 cM, 5 CM, 6 CM, 7 CM, 8 CM, 9 CM, 10 cM or larger. In other aspects, the soybean plant genomic integration site can comprise various components. Such components can include target sites. In some aspects, the soybean plant genomic integration site can comprise at least 1, 2, 3, 4, 5, 6, 7, 8, 9, 10, or more target sites. The soybean plant genomic integration site can be cleaved at the target site by a site specific nuclease. The resulting double strand break can be repaired to incorporate a donor polynucleotide that is incorporated within the genome of the soybean plant.

In an embodiment the soybean plant genomic integration site comprises low genetic diversity. The genetic diversity of a soybean plant can be determined through methods known in the art. The regions of a soybean plant genome can be categorized as either having high genetic diversity or low genetic diversity. To determine which regions of the genome are categorized as either high genetic diversity or low genetic diversity, one with skill in the art can compare homologous genomic regions among multiple soybean plant varieties. The regions of the genome that have high levels of genetic diversity will include more variability within the genomic sequences. For example, the soybean plant varieties will have numerous Single Nucleotide Polymorphisms (SNPs) within the genomic region as compared to other soybean plant varieties. Conversely, the regions of the genome that have low levels of genetic diversity will include less variability within the genomic sequences. These regions will be almost identical when comparing a genomic region across multiple soybean plant varieties. Typically the low genetic diversity regions have one dominant haplotype and may have one or more additional minor haplotypes. In an embodiment, the low genetic diversity region includes at least one major haplotype in a breeding group that is at a frequency of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In a further embodiment, the low genetic diversity region includes at least one major haplotype in a maturity group that is at a frequency of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In an additional embodiment, the low genetic diversity region includes at least one major haplotype in a haplotype and maturity group that is at a frequency of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

To assess the levels of genetic diversity in a region of a soybean plant genome, a large number of soybean plant varieties are sequenced. The resulting soybean plant genomes are then broken intobins based on genetic and physical coordinates. Other genomic features are included within the bins to differentiate the regions. For example, SNPs can be identified within the bins. Next the bins are categorized as haplotypes based on the SNP allele profiles within bins. Bins that have similar genotypes across varieties without large amounts of differentiation (e.g,. low or no occurrence of SNPs) are considered the same haplotype. In certain embodiments the bins with a major haplotype at a frequency of 80-100% across a defined germplasm comprise low diversity regions. The skilled artisan would understand that haplotype frequency can differ in different germplasm pools. The germplasm pools of these genomic regions in the soybean plant varieties may vary due to maturity differences, heterotic groupings, geographical distribution, etc.

In an embodiment the soybean plant genomic integration site comprises high recombination frequency. In an aspect, the soybean plant genomic integration site is greater than 10 cM from regions that are known to be recalcitrant to recombination. For example, pericentromeric regions are not regions of the soybean plant genome that are known to comprise high recombination frequency. In further aspects, the recombination rate of regions with high recombination frequency is greater than 0.7 cM/1 Mb.

In an embodiment the soybean plant genomic integration site comprises a polynucleotide that is in close proximity to a telomere. In some aspects the close proximity of the soybean plant genomic site is less than 20 cM from the end of a chromosome. In other aspects, the close proximity of the soybean plant genomic site is less than 20 cM, 19 cM, 18 cM, 17 cM, 16 cM, 15 cM, 14 cM, 13 cM, 12 cM, 11 cM, 10 cM, 9 cM, 8 CM, 7 cM, 6 CM, 5 CM, 4 cM, 3 CM, 2 cM, or 1 cM from the end of a chromosome. In further aspects, the close proximity of the soybean plant genomic site is less than 4.7 Mb from the end of a chromosome. In aspects, the close proximity of the soybean plant genomic site is less than 4.7 Mb, 4.6 Mb, 4.5 Mb, 4.4 Mb, 4.3 Mb, 4.2 Mb, 4.1 Mb, 4.0 Mb, 3.9 Mb, 3.8 Mb, 3.7 Mb, 3.6 Mb, 3.5 Mb, 3.4 Mb, 3.3 Mb, 3.2 Mb, 3.1 Mb, 3.0 Mb, 2.9 Mb, 2.8 Mb, 2.7 Mb, 2.6 Mb, 2.5 Mb, 2.4 Mb, 2.3 Mb, 2.2 Mb, 2.1 Mb, 2.0 Mb, 1.9 Mb, 1.8 Mb, 1.7 Mb, 1.6 Mb, 1.5 Mb, 1.4 Mb, 1.3 Mb, 1.2 Mb, 1.1 Mb, or 1.0 Mb from the end of a chromosome.

In an embodiment the soybean plant genomic integration site comprises a polynucleotide that is free of structural variation. In an aspect, the soybean plant genomic integration site does not contain a translocation. In other aspects, the soybean plant genomic integration site does not contain an inversion. In further aspects, the soybean plant genomic integration site does not contain a deletion. Those with skill in the art would appreciate that these types of structural variants could inhibit or reduce combination within the target region, or prevent efficient introgression of the target segment into another genome. As an example, the subject disclosure compared genomes from 26 public soybean genome assemblies and 10 internal soybean assemblies in the target low-diversity regions. These low diversity regions were inspected for structural variations including inversions, insertion/deletions, and translocations larger than 200 kb within 10 cM of the target low-diversity regions. Examples of identifying such structural variations are further provided in Liu, Yucheng, et al. “Pan-genome of wild and cultivated soybeans.” Cell 182.1 (2020): 162-176.

In an embodiment the soybean plant genomic integration site comprises a polynucleotide that comprises a genetic size of less than 10 cM. In some aspects the genetic size is less than 10 cM, 9 cM, 8 CM, 7 CM, 6 CM, 5 CM, 4 cM, 3 cM, 2 cM, or 1 cM. Those with skill in the art will appreciate that the genetic size is based upon genetic maps that are relative to the populations used to create the genetic map. In some aspects, a 1 cM bin used to perform the diversity analysis based on the genetic map, the resulting genetic size is determined from the analysis.

In an embodiment the plant genomic integration site comprises a polynucleotide with a genetic size of less than 10 cM. In some aspects the genetic size is less than 10 cM, 9 CM, 8 CM, 7 CM, 6 CM, 5 CM, 4 cM, 3 cM, 2 cM, or 1 cM. Those with skill in the art will appreciate that the genetic size is based upon genetic maps that are relative to the populations used to create the genetic map. In some aspects, a 1 cM bin used to perform the diversity analysis based on the genetic map, the resulting genetic size is determined from the analysis.

In an embodiment the plant genomic integration site comprises a polynucleotide with a physical size of less than 2.6 Mb. In an aspect, the soybean plant genomic integration site comprises a polynucleotide with a physical size of less than 2.6 Mb, 2.5 Mb, 2.4 Mb, 2.3 Mb, 2.2 Mb, 2.1 Mb, 2.0 Mb, 1.9 Mb, 1.8 Mb, 1.7 Mb, 1.6 Mb, 1.5 Mb, 1.4 Mb, 1.3 Mb, 1.2 Mb, 1.1 Mb, or 1.0 Mb. In other embodiments, the soybean plant genomic integration site comprises a polynucleotide with a physical size that ranges in size from 785,899 bp to 954,789 bp. In further embodiments, the soybean plant genomic integration site comprises a polynucleotide with a physical size of 785,899 bp, 790,000 bp, 795,000 bp, 800,000 bp, 805,000 bp, 810,000 bp, 815,000 bp, 820,000 bp, 825,000 bp, 830,000 bp, 835,000 bp, 840,000 bp, 845,000 bp, 850,000 bp, 855,000 bp, 860,000 bp, 865,000 bp 870,000 bp, 875,000 bp, 880,000 bp, 885,000 bp, 890,000 bp, 895,000 bp, 900,000 bp, 905,000 bp, 910,000 bp, 915,000 bp, 920,000 bp, 925,000 bp, 930,000 bp, 935,000 bp, 940,000 bp, 945,000 bp, 950,000 bp, or 954,789 bp. Those with skill in the art would appreciate that each chromosome has a physical “length” measured in base pairs (bp) starting at the first nucleotide in the genome assembly and extending to the last. In certain embodiments the physical size of the low diversity region is the approximate number of nucleotides that comprise the respective low diversity genetic bins.

In an embodiment the soybean plant genomic integration site comprises euchromatin. Euchromatin is loosely-packed, protein-bound DNA that is relatively less compact during the cell cycle. Those with skill in the art will appreciate the genomic regions comprising euchromatin undergo higher rates of recombination based on the genetic to physical relationship across the chromosome. In some aspects, the target sites of the soybean plant genomic integration site located within euchromatin are desirable for efficient recombination and Site Specific Integration (SSI) of a donor polynucleotide.

In an embodiment the soybean plant genomic integration site comprises a polynucleotide that is greater than 10 cM from heterochromatin. In other embodiments the soybean plant genomic integration site comprises a polynucleotide that is greater than 1.05 Mb from heterochromatin. Those with skill in the art will appreciate that heterochromatin is a tightly-packed, protein-bound DNA that remains compact during the cell cycle. Heterochromatin generally undergoes lower rates of recombination during meiosis. In addition, heterochromatin typically contains the centromere and pericentromeric regions. In some aspects, the target sites of the soybean plant genomic integration site located outside of a region heterochromatin are desirable for efficient recombination and Site Specific Integration (SSI) of a donor polynucleotide.

Table 1 provides the location of euchromatin and heterochromatin as disclosed in Song, Qijian, et al. “Construction of high resolution genetic linkage maps to improve the soybean genome sequence assembly Glyma1. 01.” BMC genomics 17.1 (2016): 1-11.

TABLE 1
Soybean genomic regions comprising heterochromatic
and euchromatic region.

| | Heterochromatic region (Mb) | Euchromatic region (Mb) |
|---|---|---|
| Chr01 | 8.1-47.4 | 1-8.1; 47.4-56.8 |
| Chr02 | 16.0-38.2 | 1-16.0; 38.2-48.6 |
| Chr03 | 6.9-33.4 | 1-6.9; 33.4-45.8 |
| Chr04 | 10.4-43.5 | 1-10.4; 43.5-52.4 |
| Chr05 | 6.4-30.2 | 1-6.4; 30.2-42.2 |
| Chr06 | 18.2-44.4 | 1-18.2; 44.4-51.4 |
| Chr07 | 17.7-34.6 | 1-17.7; 34.6-44.6 |
| Chr08 | 22.9-40.4 | 1-22.9; 40.4-47.8 |
| Chr09 | 6.4-38.8 | 1-6.4; 38.8-50.2 |
| Chr10 | 6.9-36.9 | 1-6.9; 36.9-51.5 |
| Chr11 | 11.4-30.0 | 1-11.4; 30.0-34.7 |
| Chr12 | 8.2-32.4 | 1-8.2; 32.4-40.0 |
| Chr13 | 0-13.3 | 1-0; 13.3-45.8 |
| Chr14 | 9.7-43.7 | 1-9.7; 43.7-49.0 |
| Chr15 | 18.3-43.0 | 1-18.3; 43.0-51.7 |
| Chr16 | 8.3-26.8 | 1-8.3; 26.8-37.8 |
| Chr17 | 14.3-35.8 | 1-14.3; 35.8-41.6 |
| Chr18 | 20.5-43.3 | 1-20.5; 43.3-58.0 |
| Chr19 | 8.9-34.3 | 1-8.9; 34.3-50.7 |
| Chr20 | 3.2-33.7 | 1-3.2; 33.7-47.9 |
| Total | 501.4 (53%) | 447.8 (47%) |

In an embodiment the soybean plant genomic integration site comprises a polynucleotide near a Quantitative Trait Loci (QTL). In some aspects the soybean plant genomic integration site is “genetically linked” to a QTL. In certain aspects the soybean plant genomic integration site is “genetically linked” to a QTL when the QTL and soybean plant genomic integration site are within at least 50 cM of one another. In other aspects the soybean plant genomic integration site is “tightly genetically linked” to a QTL. In certain aspects the soybean plant genomic integration site is “tightly genetically linked” to a QTL when the QTL and plant genomic integration site are within at least 10 cM of one another. In other aspects the soybean plant genomic integration site is “genetically linked” to a QTL, wherein the QTL and soybean plant genomic integration site are within 1 cM, 2 cM, 3 CM, 4 CM, 5 CM, 6 CM, 7 CM, 8 CM, 9 CM, 10 cM, 15 CM, 20 cM, 25 CM, 30 cM, 35 cM, 40 cM, 45 cM, of 50 cM of one another. In an embodiment the soybean plant genomic integration site comprises a polynucleotide that does not comprise an endogenous gene. In certain aspects, the plant genomic integration site comprises a polynucleotide that does not encode a coding sequence that is translated into a protein. As used herein, the terms “quantitative trait loci” and “QTL” refer to a genomic region affecting the phenotypic variation in continuously varying traits like yield or resistance. A QTL can comprise multiple genes or other genetic factors even within a contiguous genomic region or linkage group.

### Traits For Introgression

Transgenes of interest may be expressed within the genomic sequences of the of the subject disclosure. Exemplary transgenes of interest that are suitable for use in the present disclosed constructs include, but are not limited to, coding sequences that confer (1) resistance to pests or disease, (2) tolerance to herbicides, (3) value added agronomic traits, such as; yield improvement, nitrogen use efficiency, water use efficiency, and nutritional quality, (4) binding of a protein to DNA in a site specific manner, (5) expression of small RNA, and (6) selectable markers. In accordance with one embodiment, the transgene/heterologous coding sequence encoding a selectable marker or a gene product conferring insecticidal resistance, herbicide tolerance, small RNA expression, nitrogen use efficiency, water use efficiency, or nutritional quality is targeted within the genomic sequences of the subject disclosure.

### 1. Insect Resistance

Various insect resistance genes can be targeted for insertion within the genomic sequences of the subject disclosure. Regulatory elements can be engineered into a gene expression cassette containing an insect resistance gene. The operably linked sequences can then be incorporated into a chosen vector to allow for identification and selection of transformed plants (“transformants”). Exemplary insect resistance coding sequences are known in the art. As embodiments of insect resistance coding sequences that can be operably linked to the regulatory elements of the subject disclosure, the following traits are provided. Coding sequences that provide exemplary Lepidopteran insect resistance include: cry1A; cry1A.105; cry1Ab; cry1Ab (truncated); cry1Ab-Ac (fusion protein); cry1Ac (marketed as Widestrike®); cry1C; cry1F (marketed as Widestrike®); cry1Fa2; cry2Ab2; cry2Ae; cry9C; mocry1F; pinII (protease inhibitor protein); vip3A (a); and vip3Aa20. Coding sequences that provide exemplary Coleopteran insect resistance include: cry34Ab1 (marketed as Herculex®); cry35Ab1 (marketed as Herculex®); cry3A; cry3Bb1; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

### 2. Herbicide Tolerance

Various herbicide tolerance genes can be targeted for insertion within the genomic sequences of the subject disclosure. Regulatory elements can be engineered into a gene expression cassette containing a herbicide tolerance gene. The operably linked sequences can then be incorporated into a chosen vector to allow for identification and selection of transformed plants (“transformants”). Exemplary herbicide tolerance coding sequences are known in the art. As embodiments of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include: mutant EPSPS genes such as 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). These traits are currently marketed as Gly-Tol™, Optimum® GAT®, Agrisure® GT and Roundup Ready®. Resistance genes for glufosinate and/or bialaphos compounds include dsm-2, bar and pat genes. The bar and pat traits are currently marketed as LibertyLink®. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF_W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Acc1-S1, Acc1-S2 and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), Is+ genes (tolerance to triazine), and nitrilase genes (tolerance to benzonitrile). The above list of herbicide tolerance genes is not meant to be limiting. Any herbicide tolerance genes are encompassed by the present disclosure.

### 3. Agronomic Traits

Various agronomic trait genes can be targeted for insertion within the genomic sequences of the subject disclosure. Regulatory elements can be engineered into a gene expression cassette containing an agronomic trait gene. The operably linked sequences can then be incorporated into a chosen vector to allow for identification and selection of transformed plants (“transformants”). Exemplary agronomic trait coding sequences are known in the art. As embodiments of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

### 4. DNA Binding Proteins

Various DNA binding transgene/heterologous coding sequences can be targeted for insertion within the genomic sequences of the subject disclosure. Regulatory elements can be engineered into a gene expression cassette containing a DNA binding gene. The operably linked sequences can then be incorporated into a chosen vector to allow for identification and selectable of transformed plants (“transformants”). Exemplary DNA binding protein coding sequences are known in the art. As embodiments of DNA binding protein coding sequences that can be operably linked to the regulatory elements of the subject disclosure, the following types of DNA binding proteins can include; Zinc Fingers, TALENS, CRISPRS, and meganucleases. The above list of DNA binding protein coding sequences is not meant to be limiting. Any DNA binding protein coding sequences is encompassed by the present disclosure.

### 5. Small RNA

Various small RNA sequences can be targeted for insertion within the genomic sequences of the subject disclosure. Regulatory elements can be engineered into a gene expression cassette containing a small RNA sequence. The operably linked sequences can then be incorporated into a chosen vector to allow for identification and selection of transformed plants (“transformants”). Exemplary small RNA traits are known in the art. As embodiments of small RNA coding sequences that can be operably linked to the regulatory elements of the subject disclosure, the following traits are provided. For example, delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the Black Spot Bruise Tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pRI small RNA (degrades R1 transcripts to limit the formation of reducing sugars through starch degradation). Additional, benefits such as reduced acrylamide resulting from the asnl small RNA that triggers degradation of Asnl to impair asparagine formation and reduce polyacrylamide. Finally, the non-browning phenotype of pgas ppo suppression small RNA results in suppressing PPO to produce apples with a non-browning phenotype. The above list of small RNAs is not meant to be limiting. Any small RNA encoding sequences are encompassed by the present disclosure.

### 6. Selectable Markers

Various selectable markers also described as reporter genes can be targeted for insertion within the genomic sequences of the subject disclosure. Regulatory elements can be engineered into a gene expression cassette containing a reporter gene. The operably linked sequences can then be incorporated into a chosen vector to allow for identification and selectable of transformed plants (“transformants”). Many methods are available to confirm expression of selectable markers in transformed plants, including for example DNA sequencing and PCR (polymerase chain reaction), Southern blotting, RNA blotting, immunological methods for detection of a protein expressed from the vector. But, usually the reporter genes are observed through visual observation of proteins that when expressed produce a colored product. Exemplary reporter genes are known in the art and encode β-glucuronidase (GUS), luciferase, green fluorescent protein (GFP), yellow fluorescent protein (YFP, Phi-YFP), red fluorescent protein (DsRFP, RFP, etc), β-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for selection of transformed cells or tissues. Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

In an embodiment, herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridochromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Acc1-S1, Acc1-S2 and Acc1-S3. In an embodiment, herbicides can inhibit photosynthesis, including triazine (psbA and 1s+ genes) or benzonitrile (nitrilase gene). Furthermore, such selectable markers can include positive selection markers such as phosphomannose isomerase (PMI) enzyme.

In an embodiment, selectable marker genes include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). An embodiment also includes selectable marker genes encoding resistance to: chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

In some embodiments the coding sequences are synthesized for optimal expression in a soybean plant. For example, in an embodiment, a coding sequence of a gene has been modified by codon optimization to enhance expression in soybean plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. In an embodiment, a coding sequence, gene, heterologous coding sequence or transgene/heterologous coding sequence is designed to be expressed in soybean plants at a higher level resulting in higher transformation efficiency. Methods for plant optimization of genes are well known. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

### Molecular Confirmation

Methods of confirming the presence of a gene expression cassette within the genome of a soybean plant are known in the art. For example the detection of a gene expression cassette within the genome of a soybean plant can be achieved, for example, by the polymerase chain reaction (PCR). The PCR detection is performed with the use of two oligonucleotide primers flanking the polymorphic segment of the polymorphism followed by DNA amplification. This step involves repeated cycles of heat denaturation of the DNA followed by annealing of the primers to their complementary sequences at low temperatures, and extension of the annealed primers with DNA polymerase. Size separation of DNA fragments on agarose or polyacrylamide gels following amplification, comprises the major part of the methodology. Such selection and screening methodologies are well known to those skilled in the art. Molecular confirmation methods that can be used to identify plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay, otherwise known as TAQMAN® (Life Technologies, Foster City, Calif.), is a method of detecting and quantifying the presence of a DNA sequence. Briefly, a FRET oligonucleotide probe is designed with one portion of the oligo within the transgene and the other portion in the flanking genomic sequence for event-specific detection. The FRET probe and PCR primers (one primer in the insert DNA sequence and one in the flanking genomic sequence) are cycled in the presence of a thermostable polymerase and dNTPs. During PCR amplification the 5′-3′ exonuclease activity of Taq polymerase results in the hydrolysis of the FRET probe bound to the target sequence, which in turn releases the quenched fluorophore and allows detection of a fluorescent signal. A fluorescent signal indicates the presence of the flanking/transgene insert sequence due to successful amplification and hybridization. Such a hydrolysis probe assay for detection of as an amplification reaction is an embodiment of the subject disclosure.

KASPar® assays are a method of detecting and quantifying the presence of a DNA sequence. Briefly, the genomic DNA sample comprising the integrated gene expression cassette polynucleotide is screened using a polymerase chain reaction (PCR) based assay known as a KASPar® assay system. The KASPar® assay used in the practice of the subject disclosure can utilize a KASPar® PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the KASPar® PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence. Such a KASPar® assay for detection of an amplification reaction is an embodiment of the subject disclosure.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed, such as YO-PRO-1®, SYTOX Green®, SYBR Green IR, SYTO11®, SYTO12®, SYTO13®, BOBO®, YOYO®, and TOTO®.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction. Three NGS platforms are commercially available, the Genome Sequencer FLX™ from 454 Life Sciences/Roche, the Illumina Genome Analyser™ from Solexa and Applied Biosystems' SOLiD™ (acronym for: ‘Sequencing by Oligo Ligation and Detection’). In addition, there are two single molecule sequencing methods available. These include the true Single Molecule Sequencing (tSMS) from Helicos Bioscience™ and the Single Molecule Real Time™ sequencing (SMRT) from Pacific Biosciences.

The Genome Sequencher FLX™ which is marketed by 454 Life Sciences/Roche is a long read NGS, which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The Illumina Genome Analyser™ which is marketed by Solexa™ is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The Sequencing by Oligo Ligation and Detection (SOLID) system marketed by Applied Biosystems™ is a short read technology. This NGS technology uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

The tSMS of Helicos Bioscience™ and SMRT of Pacific Biosciences™ apply a different approach which uses single DNA molecules for the sequence reactions. The tSMS Helicos™ system produces up to 800 million short reads that result in 21 gigabases per run. These reactions are completed using fluorescent dye-labelled virtual terminator nucleotides that is described as a ‘sequencing by synthesis’ approach.

The SMRT Next Generation Sequencing system marketed by Pacific Biosciences™ uses a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

### Plants

In an embodiment a soybean plant, plant tissue, or plant cell comprises a donor polynucleotide integrated within the genomic sequence of the subject disclosure. In one embodiment a soybean plant, plant tissue, plant part, or plant cell comprises the genomic sequence of the subject disclosure or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:206 or SEQ ID NO:207. In an embodiment a soybean plant, plant tissue, plant part, or plant cell comprises a donor polynucleotide integrated within the genomic sequence of the subject disclosure. In one embodiment a soybean plant, plant tissue, plant part, or plant cell comprises the genomic sequence of the subject disclosure or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:1-109 or SEQ ID NO:264-334. In an embodiment, a soybean plant, plant tissue, plant part, or plant cell comprises a gene expression cassette integrated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:206 or SEQ ID NO:207, wherein the donor polynucleotide is operably linked to a transgene or heterologous coding sequence is an insecticidal resistance transgene, an herbicide tolerance transgene, a nitrogen use efficiency transgene, a water use efficiency transgene, a nutritional quality transgene, a DNA binding transgene, a selectable marker transgene, or combinations thereof. In an embodiment, a soybean plant, plant tissue, or plant cell comprises a gene expression cassette integrated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:1-109 or SEQ ID NO:264-334, wherein the donor polynucleotide is operably linked to a transgene or heterologous coding sequence is an insecticidal resistance transgene, an herbicide tolerance transgene, a nitrogen use efficiency transgene, a water use efficiency transgene, a nutritional quality transgene, a DNA binding transgene, a selectable marker transgene, or combinations thereof.

One of skill in the art will recognize that after the exogenous donor polynucleotide is stably incorporated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:206 or SEQ ID NO:207 of a plant and confirmed to be operable, it can be introduced into other soybean plants by sexual crossing. In a further embodiment one of skill in the art will recognize that after the exogenous donor polynucleotide is stably incorporated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:1-109 or SEQ ID NO:264-334 of a plant and confirmed to be operable, it can be introduced into other soybean plants by sexual crossing. Any number of standard breeding techniques can be used, depending upon the species to be crossed.

The present disclosure also encompasses seeds of the soybean plants described above, wherein the seed has the transgene/heterologous coding sequence or gene construct containing the gene regulatory elements of the subject disclosure. The present disclosure further encompasses the progeny, clones, cell lines or cells of the soybean plants described above wherein said progeny, clone, cell line or cell has the transgene/heterologous coding sequence or gene construct containing the gene regulatory elements of the subject disclosure.

The present disclosure also encompasses the cultivation of soybean plants described above, wherein the soybean plant has the transgene/heterologous coding sequence or gene construct containing the donor polynucleotide integrated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:206 or SEQ ID NO:207. In another embodiment the disclosure also encompasses the cultivation of soybean plants described above, wherein the soybean plant has the transgene/heterologous coding sequence or gene construct containing the donor polynucleotide integrated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:1-109 or SEQ ID NO:264-334. Accordingly, such soybean plants may be engineered to, inter alia, have one or more desired traits or events containing gene regulatory elements, by being transformed with nucleic acid molecules according to the invention, and may be cropped or cultivated by any method known to those of skill in the art.

### Method of Expressing a Gene

In an embodiment, a method of expressing at least one transgene/heterologous coding sequence in a soybean plant comprises growing a soybean plant comprising a donor polynucleotide integrated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:206 or SEQ ID NO:207. In an embodiment, a method of expressing at least one transgene/heterologous coding sequence in a soybean plant comprises growing a soybean plant comprising a donor polynucleotide integrated within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO: 1-109 or SEQ ID NO:264-334. In an embodiment, a method of expressing at least one transgene/heterologous coding sequence in a soybean plant tissue or soybean plant cell comprises culturing a soybean plant tissue or soybean plant cell comprising a transgene/heterologous coding sequence that is integrated as a donor polynucleotide within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:206 or SEQ ID NO:207. In an embodiment, a method of expressing at least one transgene/heterologous coding sequence in a soybean plant tissue or soybean plant cell comprises culturing a soybean plant tissue or soybean plant cell comprising a transgene/heterologous coding sequence that is integrated as a donor polynucleotide within the genomic sequence of the subject disclosure, or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:1-109 or SEQ ID NO:264-334.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

Embodiments of the subject disclosure are further exemplified in the following Examples. It should be understood that these Examples are given by way of illustration only. From the above embodiments and the following Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The following is provided by way of illustration and not intended to limit the scope of the invention.

In an embodiment a soybean plant, plant tissue, or plant cell comprises a donor polynucleotide integrated within the genomic sequence of the subject disclosure. In one embodiment a soybean plant, plant tissue, or plant cell comprises the genomic sequence of the subject disclosure or a sequence that has at least 80%, 81%, 82%, 83%, 84%, 85%, 86%, 87%, 88%, 89%, 90%, 91%, 92%, 93%, 94%, 95%, 96%, 97%, 98%, 99% or 99.5% sequence identity with a sequence selected from SEQ ID NO:206 or SEQ ID NO:207.

### EXAMPLES

### Example 1: Identification of Genomic Integration Sites

Traditional transformation methods using random integration of exogenous DNA will produce material with transgenes in genomic sites that are not optimal for downstream plant breeding applications. During subsequent introgression of these transgene into elite germplasm, undesirable linked DNA from a donor is incorporated (for example, linkage drag) that may negatively impact agriculturally important traits, such as yield and biomass (Peng, T., Sun, X. & Mumm, R. H. Optimized breeding strategies for multiple trait integration: I. Minimizing linkage drag in single event introgression. Mol Breeding 33, 89-104 (2014)). To mitigate this risk and improve breeding efficiencies, it is desirable to place transgenes in regions that minimally impact performance when the transgene and surrounding genomic region is introgressed into diverse elite germplasm.

The subject disclosure provides site-specific integration strategies that place transgenes in favorable genomic regions where the genomic integration sites comprises the following characteristics:
1. Low genetic diversity across a target germplasm,
2. High expected recombination frequencies,
3. Absence of large structural variation in the immediate region, and
4. Near telomeres and away from pericentromeric regions.

Once a desirable genomic integration site is selected, specific transformation target sites within the genomic region can be identified and refined based on their proximity to known genes and regulatory regions and the presence of sequence features required for integration.

At the genomic level, genetic diversity is assessed by the presence and frequency of genomic variants, such as single nucleotide polymorphisms (SNPs), across individuals within a population. While some regions of the genome are necessarily variable and drive phenotypic variation, other regions exist where diversity is limited or absent. Presumptively, regions that are identical in DNA sequence would have minimal impact on phenotype and genotype when moved between two genetically distinct individual plants. These regions are desirable as genomic integration sites as they would minimize the effects of linkage drag during introgression.

To simplify genetic diversity assessments, a genome of a plant variety can be broken into bins based on genetic or physical coordinates and groups of SNPs. Typically the bins are greater than or equal to 1 cM. Next, the bin can be analyzed and compared to bins of the other plant varieties for assignments as haplotypes. The bins that share a similar SNP profile within a bin are considered to have the same haplotype. The frequency of each haplotype across a target germplasm can then be calculated; those bins with no haplotype differentiation or the bins with high frequency of a single haplotype can be identified as low diversity regions.

To facilitate efficient transgene introgression strategies, it is ideal to select low diversity regions that are large in genetic size (e.g., >1 cM) while avoiding genomic segments recalcitrant to recombination, such as in pericentromeric regions where physical to genetic distance ratios are relatively large. Additionally, it is desired to avoid regions of the genome where large structural variants (said structural variations include translocations, inversions, deletions) may impact recombination near a target site. Target regions can be further refined based on their proximity to known QTL, gene density in the region, and methylation levels. These features make up the presence of genomic sequence features needed for site-specific integration.

Transgenes integrated within genomic integration sites that possess the characteristics as described above, allow researchers to enact efficient introgression strategies that minimize negative donor linkage drag. These regions are also ideal for strategies aimed at stacking multiple transgenic loci in a single region to form complex trait loci (Gao, H., Mutti, J., Young, J. K., Yang, M., Schroder, M., Lenderts, B., & Feigenbutz, L. (2020). Complex Trait Loci in Maize Enabled by CRISPR-Cas9 Mediated Gene Insertion. Frontiers in Plant Science, 11, 535.).

### Example 2: Genomic Integration Site Criteria

The following methodology are used to identify and obtain specific genomic integration sites.

Step 1: Plant varieties are genotyped using re-sequencing or through high-density SNP genotyping platform to genotype varieties representing the diversity within a closed breeding program.

Step 2: The obtained genotyping information is used to create a haplotype framework based on 1 cM genetic bins spanning the entire genome (Coffman, S. M., Hufford, M. B., Andorf, C. M., & Lübberstedt, T. (2020). Haplotype structure in commercial maize breeding programs in relation to key founder lines. Theoretical and Applied Genetics, 133 (2), 547-561.). The genetic bins can be various sizes depending on the application and may range in size from 5775 bp to 20,616,638 bp.

Step 3: The genetic diversity is assessed across the genome within the germplasm pool. For example, haplotype frequencies based on predefined haplotypes created from high-density SNP profiles within 1 cM genetic bins are used to assess the genetic diversity. Other reliable diversity metrics include-nucleotide diversity (Nei and Lei, Oct. 1, 1979 “Mathematical Model for Studying Genetic Variation in Terms of Restriction Endonucleases”. PNAS. 76 (10): 5269-73.) and haplotype diversity (Nei M (1987) Molecular evolutionary genetics. Columbia University Press, Columbia).

Step 4: Next the genome is analyzed to identify regions that possess low diversity, hereinafter low diversity regions. These types of regions are 1 cM in length or greater and the major haplotype is at 80-100% frequency. The low diversity region may range in size from 5 kb to 2100 kb.

Step 5: the low diversity regions are assessed for favorable breeding characteristics. For example, a low diversity region is ideally located in in a genomic segment where gene density is low and genes are constitutively expressed. Furthermore, such a low diversity region will not be co-located or linked to a large structural variant such as a translocation, deletion, or inversion. These types of large structural variants could inhibit or reduce recombination within the target region, or prevent efficient introgression of the target segment into another genome. Additionally, the low diversity region is greater than 10 cM from genomic regions that are recalcitrant to recombination. Such regions that are recalcitrant to recombination are often marked by a low genetic to physical distance ratio. These types of genomic regions are correlated with pericentromeric regions and tend to have high repeat content. Conversely, the low diversity region will occur near a telomere of the chromosome, where recombination rates tend to be higher and trait introgression may be simplified. Exemplary regions of a low diversity region include Chr02:4-9 cM (1,169,882-2,124,671 bp) and Chr01:13-20 cM (1,691,001-2,476,900) of the Williams82 genomic assembly, w82.a2.v1.

These genomic integration sites are novel sequences that possess superior qualities as regions for targeted insertion of a polynucleotide fragment.

### Example 3: Ideal Target Site Analysis

Whole genome shotgun sequencing libraries were prepared for 45 varieties of soybean according to standard sequencing protocols. Libraries were sequenced on the Illumina HiSeq 2000 instrument to a depth of 15-41× sequencing depth. Sequence reads were aligned to the Soybean Genomic Assembly w82.a2.v1 by bowtie2 and compressed binary alignment/map (bam) files were generated from the alignments by SAMtools. SNPs were identified considering only unique read alignments, where SNP sites were required to have a minimum coverage of three reads for >=50% of sequenced lines and a mean purity of >=0.98. Further curation of SNP sites was performed by discarding those with high levels of missing genotypes to produce a core set of SNPs.

An additional 1,693 varieties of soybean were sequenced using low-pass Illumina sequencing and genotypes were scored by examining read alignments at core SNP sites. Following this, missing genotypes across all lines were imputed using FILLIN (Bradbury et al., 2007 TASSEL: software for association mapping of complex traits in diverse samples, Bioinformatics, Volume 23, Issue 19, 1 Oct. 2007, Pages 2633-2635). Haplotypes were identified and scored for all samples across 1 cM bins following methods described in Coffman et al. 2020 Haplotype structure in commercial maize breeding programs in relation to key founder lines. Theoretical and Applied Genetics, 133 (2), 547-561. Centimorgan distance was calculated from a consensus genetic map adapted from Song et al. 2016 Construction of high resolution genetic linkage maps to improve the soybean genome sequence assembly Glyma1. 01. BMC genomics, 17 (1), 1-11.

Haplotype frequencies were calculated across the 1,738 soybean varieties for each 1 cM bin. The bins where the major haplotype was at greater than 80% frequency were identified and annotated as low diversity bins. The low diversity bins were assessed using the criteria listed in Example 1 and low diversity regions with ideal characteristics for site specific integration were identified.

### Example 4: Ideal Target Site, Chr01:13-20 cM (1,691,001-2,476,900) SEQ ID NO:206

A region on Chr1 extending from approximately 1,691,001 to 2,476,900 bp (13-20 cM) has desirable characteristics for exogenous DNA insertion. The major haplotype across the 8 cM region ranges from 80% to 95% frequency. Furthermore, the region is unlinked to major breeding target gene sequences, is within a region of high recombination, is greater than 20 cM from presumed heterochromatin regions, and is not linked to known major structural variation based on 26 reference soybean assemblies (Liu et al. 2020 Pan-genome of wild and cultivated soybeans. Cell, 182 (1), 162-176). Table 2 provides a description of the characteristics and physical attributes of Chr01:13-20.

TABLE 2
Chr01: 13-20 physical attributes.

| Region | Chr01: 13-20 | |
|---|---|---|
| Genetic size | 8 | cM |
| Physical size | 785,899 | bp |

| | |
|---|---|
| Near Telomere (<20 cM) | Yes |
| Euchromatin | Yes |
| Near Heterochromatin (<10 cM) | No (>20 cM) |
| Haplotype (Low) | 80% frequency |
| Haplotype (High) | 95% frequency |
| Near Major QTL | No |
| Structural Variation | No |

### Example 5: Ideal Target Site, Chr02:4-9 cM (1,169,882-2,124,671 bp) SEQ ID NO:207

A region on Chr2 extending from approximately 1,169,882-2, 124,671 bp (4-9 cM) also has desirable characteristics for exogenous DNA insertion. The major haplotype across the 6 cM region ranges from 92% to 97% frequency. Furthermore, the region is unlinked to major breeding target gene sequences, is within a region of high recombination, is greater than 70 cM from presumed heterochromatin regions, is within 10 cM of the chr02 telomere, and is not linked to known major structural variation based on 26 reference soybean assemblies (Liu et al. 2020 Pan-genome of wild and cultivated soybeans. Cell, 182 (1), 162-176). Table 3 provides a description of the characteristics and physical attributes of Chr02:4-9.

TABLE 3
Chr02: 4-9 physical attributes.

| Region | Chr02: 4-9 | |
|---|---|---|
| Genetic size | 6 | cM |
| Physical size | 954,789 | bp |

| | |
|---|---|
| Near Telomere (<20 cM) | Yes |
| Euchromatin | Yes |
| Near Heterochromatin (<10 cM) | No (>70 cM) |
| Haplotype (Low) | 92% frequency |
| Haplotype (High) | 97% frequency |
| Near Major QTL | No |
| Structural Variation | No |

### Example 6: Nonideal Target Site, Chr01:48-54 (7,681,992-35,921,482 bp)

A region on Chr1 extending from 7,681,992-35,921,482 bp (48-54 cM) lacks desirable characteristics for exogenous DNA insertion. This region comprises the centromeric and pericentromeric region of Chr1 (Schmutz et al. 2010 Genome sequence of the palaeopolyploid soybean. nature, 463 (7278), 178-183). It is a region of low recombination rates marked by an average of 4,034,213 bp/1 cM. Hence, introgression of a transgene from this region into different genetic backgrounds would often lead to progeny carrying large segments of flanking DNA from the transgene donor. Additionally, pericentromeric regions tend to have lower levels of gene expression and contain a high number of repetitive elements (Du et al. 2012 Pericentromeric effects shape the patterns of divergence, retention, and expression of duplicated genes in the paleopolyploid soybean. The Plant Cell, 24 (1), 21-32), and therefore, genes inserted in this region may have inhibited or reduced expression.

Within the studied germplasm, this region does not meet the “low-diversity” criteria as the major haplotype frequency ranges from 58-62%—a characteristic that would limit haplotype matches across the region between donor and non-donors and increase the chances that negative linkage drag would occur during breeding. Finally, large structural variation has been discovered across this genomic region on chromosome 1 that could reduce local recombination, and thus diminish efficient introgression of a transgene located near it, when parents used in a cross are polymorphic for the variant sites (Liu et al. 2020 Pan-genome of wild and cultivated soybeans. Cell, 182 (1), 162-176).

### Example 7: Selection of Soy Genomic Windows for the Introduction of SSI Target Sites by the Guide RNA/Cas Endonuclease System and Complex Trait Loci Development

To develop a Complex Trait Locus in the soy genome, a method was developed to introduce transgenic SSI (Site Specific Integration) target sites in a soy genomic locus of interest using the guide RNA/Cas9 or Cas12fl endonuclease system. First, a genomic window was identified into which multiple SSI target sites (CRISPR-Cas9 or Cas12fl sites) in proximity can be introduced. Several chromosome regions were identified, two soy genomic regions (also referred to as genomic windows) were selected to produce Complex Trait Loci following diversity analysis as described in the previous Examples. The first soy genomic window for development of a Complex Trait Locus (CTL) spans from Chr01:1,730,000 to Chr01:2,513,000 on chromosome 1. Streptococcus pyogenes Cas endonuclease target sites (52 sites) within the genomic window were identified as sites which are at least 2-2.5 kb away from any known gene's transcription start site and at least 500 bp, preferably over 1 kb or 2 kb away from repetitive sequences. Table 4 shows the physical and genetic map position of the sites (Ganal, M. et al (2011) PloS one, DOI: 10.1371).

TABLE 4
Genomic window comprising a Complex Trait
Locus (CTL1) on chromosome 1 of soybean.

| Name of Cas | Cas9 target | PAM Physical | Genetic | PAM Physical |
|---|---|---|---|---|
| endonuclease | site SEQ ID | position | Position | position |
| target site | NO | (93Y21) | (cM) | (w82.a2.v1) |
| Chr01-TS1 | 1 | 1743522 | 13.21 | 1703125 |
| Chr01-TS2 | 2 | 1746967 | 13.28 | 1706570 |
| Chr01-TS3 | 3 | 1758148 | 13.48 | 1717674 |
| Chr01-TS4 | 4 | 1758155 | 13.48 | 1717681 |
| Chr01-TS5 | 5 | 1758158 | 13.48 | 1717684 |
| Chr01-TS6 | 6 | 1758195 | 13.48 | 1717721 |
| Chr01-TS7 | 7 | 1769453 | 13.68 | 1728976 |
| Chr01-TS8 | 8 | 1770479 | 13.70 | 1730002 |
| Chr01-TS9 | 9 | 1801135 | 14.16 | 1758678 |
| Chr01-TS10 | 10 | 1801440 | 14.17 | 1758983 |
| Chr01-TS11 | 11 | 1802725 | 14.18 | 1760268 |
| Chr01-TS12 | 12 | 1806360 | 14.23 | 1763902 |
| Chr01-TS13 | 13 | 1806364 | 14.23 | 1763906 |
| Chr01-TS14 | 14 | 1806373 | 14.23 | 1763915 |
| Chr01-TS15 | 15 | 1837156 | 14.62 | 1794708 |
| Chr01-TS16 | 16 | 1837172 | 14.62 | 1794724 |
| Chr01-TS17 | 17 | 1837263 | 14.62 | 1794815 |
| Chr01-TS18 | 18 | 1837616 | 14.63 | 1795168 |
| Chr01-TS19 | 19 | 1837629 | 14.63 | 1795181 |
| Chr01-TS20 | 20 | 1837637 | 14.63 | 1795189 |
| Chr01-TS21 | 21 | 1837657 | 14.63 | 1795209 |
| Chr01-TS22 | 22 | 1903457 | 15.47 | 1861634 |
| Chr01-TS23 | 23 | 1904919 | 15.49 | 1863096 |
| Chr01-TS24 | 24 | 1917783 | 15.65 | 1875959 |
| Chr01-TS25 | 25 | 1942244 | 16.03 | 1900422 |
| Chr01-TS26 | 26 | 1944695 | 16.06 | 1902873 |
| Chr01-TS27 | 27 | 1944777 | 16.07 | 1902955 |
| Chr01-TS28 | 28 | 2097350 | 17.27 | 2058947 |
| Chr01-TS29 | 29 | 2127365 | 17.29 | 2089243 |
| Chr01-TS30 | 30 | 2127383 | 17.29 | 2089261 |
| Chr01-TS31 | 31 | 2172486 | 17.79 | 2134385 |
| Chr01-TS32 | 32 | 2172540 | 17.79 | 2134439 |
| Chr01-TS33 | 33 | 2174063 | 17.81 | 2135962 |
| Chr01-TS34 | 34 | 2174084 | 17.81 | 2135983 |
| Chr01-TS35 | 35 | 2174093 | 17.81 | 2135992 |
| Chr01-TS36 | 36 | 2174096 | 17.81 | 2135995 |
| Chr01-TS37 | 37 | 2262609 | 18.89 | 2223439 |
| Chr01-TS38 | 38 | 2262629 | 18.89 | 2223459 |
| Chr01-TS39 | 39 | 2263922 | 18.91 | 2228268 |
| Chr01-TS40 | 40 | 2292033 | 19.33 | 2256494 |
| Chr01-TS41 | 41 | 2292055 | 19.33 | 2256516 |
| Chr01-TS42 | 42 | 2297008 | 19.41 | 2261469 |
| Chr01-TS43 | 43 | 2355586 | 20.07 | 2320000 |
| Chr01-TS44 | 44 | 2356651 | 20.08 | 2321065 |
| Chr01-TS45 | 45 | 2389073 | 20.40 | 2353485 |
| Chr01-TS46 | 46 | 2412513 | 20.60 | 2376929 |
| Chr01-TS47 | 47 | 2412514 | 20.60 | 2376930 |
| Chr01-TS48 | 48 | 2412863 | 20.61 | 2377279 |
| Chr01-TS49 | 49 | 2481285 | 20.80 | 2445704 |
| Chr01-TS50 | 50 | 2481726 | 20.81 | 2446145 |
| Chr01-TS51 | 51 | 2481740 | 20.81 | 2446159 |
| Chr01-TS52 | 52 | 2483848 | 20.82 | 2448268 |

The second soy genomic window that was selected for development of a Complex Trait Locus (CTL) spans from Chr02:1,117,000 to Chr02:2,129,999 on chromosome 2. Streptococcus pyogenes Cas endonuclease target sites (58 sites) within the genomic window were identified as sites which are at least 2-2.5 kb away from any known gene's transcription start site and at least 500 bp preferably 1 kb or 2 kb away from repetitive sequences. Table 5 shows the physical and genetic map position of the sites (Ganal, M. et al (2011) PloS one, DOI: 10.1371).

TABLE 5
Genomic window comprising a Complex Trait
Locus 2 on chromosome 2 of soybean.

| Name Cas | | PAM Physical | Genetic | PAM Physical |
|---|---|---|---|---|
| endonuclease | Cas9 target | position | Position | position |
| target site | SEQ ID NO | (93Y21) | (cM) | (w82.a2.v1) |
| Chr02-TS1 | 53 | 1,193,409 | 3.99 | 1,165,660 |
| Chr02-TS2 | 54 | 1,194,819 | 4.00 | 1,167,070 |
| Chr02-TS3 | 55 | 1,220,694 | 4.07 | 1,192,947 |
| Chr02-TS4 | 56 | 1,234,962 | 4.11 | 1,207,215 |
| Chr02-TS5 | 57 | 1,237,306 | 4.12 | 1,209,559 |
| Chr02-TS6 | 58 | 1,370,895 | 5.04 | 1,344,254 |
| Chr02-TS7 | 59 | 1,370,919 | 5.04 | 1,344,278 |
| Chr02-TS8 | 60 | 1,372,628 | 5.05 | UNKNOWN |
| Chr02-TS9 | 61 | 1,398,930 | 5.22 | 1,380,479 |
| Chr02-TS10 | 62 | 1,438,260 | 5.46 | 1,419,803 |
| Chr02-TS11 | 63 | 1,438,275 | 5.46 | 1,419,818 |
| Chr02-TS12 | 64 | 1,438,279 | 5.46 | 1,419,822 |
| Chr02-TS13 | 65 | 1,598,311 | 7.13 | 1,587,139 |
| Chr02-TS14 | 66 | 1,598,341 | 7.13 | 1,587,169 |
| Chr02-TS15 | 67 | 1,641,537 | 7.40 | 1,630,347 |
| Chr02-TS16 | 68 | 1,643,490 | 7.42 | 1,632,300 |
| Chr02-TS17 | 69 | 1,648,891 | 7.45 | 1,637,702 |
| Chr02-TS18 | 70 | 1,648,960 | 7.45 | 1,637,771 |
| Chr02-TS19 | 71 | 1,675,336 | 7.62 | 1,664,066 |
| Chr02-TS20 | 72 | 1,678,616 | 7.64 | 1,667,338 |
| Chr02-TS21 | 73 | 1,679,657 | 7.64 | 1,668,361 |
| Chr02-TS22 | 74 | 1,700,916 | 7.78 | 1,689,608 |
| Chr02-TS23 | 75 | 1,713,028 | 7.85 | 1,701,727 |
| Chr02-TS24 | 76 | 1,714,888 | 7.86 | 1,703,587 |
| Chr02-TS25 | 77 | 1,743,108 | 8.04 | 1,731,690 |
| Chr02-TS26 | 78 | 1,743,109 | 8.04 | 1,731,691 |
| Chr02-TS27 | 79 | 1,743,147 | 8.04 | 1,731,729 |
| Chr02-TS28 | 80 | 1,761,059 | 8.15 | 1,749,738 |
| Chr02-TS29 | 81 | 1,761,068 | 8.15 | 1,749,747 |
| Chr02-TS30 | 82 | 1,761,072 | 8.15 | 1,749,751 |
| Chr02-TS31 | 83 | 1,761,073 | 8.15 | 1,749,752 |
| Chr02-TS32 | 84 | 1,772,297 | 8.22 | 1,760,962 |
| Chr02-TS33 | 85 | 1,772,300 | 8.22 | 1,760,965 |
| Chr02-TS34 | 86 | 1,772,301 | 8.22 | 1,760,966 |
| Chr02-TS35 | 87 | 1,789,946 | 8.33 | 1,778,620 |
| Chr02-TS36 | 88 | 1,798,611 | 8.39 | 1,790,217 |
| Chr02-TS37 | 89 | 1,801,537 | 8.40 | 1,793,127 |
| Chr02-TS38 | 90 | 1,801,544 | 8.40 | 1,793,134 |
| Chr02-TS39 | 91 | 1,802,145 | 8.41 | 1,793,735 |
| Chr02-TS40 | 92 | 1,802,146 | 8.41 | 1,793,736 |
| Chr02-TS41 | 93 | 1,803,499 | 8.42 | 1,795,089 |
| Chr02-TS42 | 94 | 1,918,831 | 8.87 | 1,910,560 |
| Chr02-TS43 | 95 | 1,960,668 | 9.01 | 1,952,397 |
| Chr02-TS44 | 96 | 1,990,072 | 9.15 | 1,985,431 |
| Chr02-TS45 | 97 | 1,990,238 | 9.15 | 1,985,597 |
| Chr02-TS46 | 98 | 1,990,244 | 9.15 | 1,985,603 |
| Chr02-TS47 | 99 | 1,990,248 | 9.15 | 1,985,607 |
| Chr02-TS48 | 100 | 1,991,782 | 9.16 | 1,987,150 |
| Chr02-TS49 | 101 | 1,991,799 | 9.16 | 1,987,167 |
| Chr02-TS50 | 102 | 1,998,982 | 9.21 | 1,994,347 |
| Chr02-TS51 | 103 | 2,002,443 | 9.23 | 1,997,808 |
| Chr02-TS52 | 104 | 2,002,487 | 9.23 | 1,997,852 |
| Chr02-TS53 | 105 | 2,002,488 | 9.23 | 1,997,853 |
| Chr02-TS54 | 106 | 2,008,351 | 9.27 | 2,003,716 |
| Chr02-TS55 | 107 | 2,010,996 | 9.29 | 2,006,437 |
| Chr02-TS56 | 108 | 2,029,969 | 9.41 | 2,025,428 |
| Chr02-TS57 | 109 | 2,081,706 | 9.74 | 2,077,352 |

### Example 8: DNA Constructs of the Guide RNA/Cas Endonuclease System for Genome Modifications in Soy

To achieve best expression and highest activity for guide RNA/Cas endonuclease system in soy, the Cas9 gene from Streptococcus pyogenes M1 GAS (SF370) (SEQ ID NO: 110) was soy codon optimized per standard techniques known in the art and the potato ST-LS1 intron was introduced in order to eliminate its expression in E. coli and Agrobacterium. To facilitate nuclear localization of the Cas9 protein in soy cells, Arabidopsis thaliana nuclear localization signal AT-NLS (SEQ ID NO: 111) and Agrobacterium tumefaciens bipartite VirD2 T-DNA border endonuclease carboxyl terminal nuclear localization signal (SEQ ID NO: 112) were incorporated at the amino and carboxyl-termini of the Cas9 open reading frame respectively. The soy optimized Cas9 gene was operably linked to a soy elongation factor (EF1A2) promoter by standard molecular biological techniques. The soy U6-13.1 polymerase III promoter was used to express guide RNAs which direct Cas9 nuclease to designated genomic sites (U.S. patent application No. 20180230476). The guide RNA coding sequence was 77 bp long and comprised a 20 bp variable targeting domain from a chosen soy genomic target site on the 5′ end soy U6 polymerase III terminator (U.S. patent application No. 20180230476). The variable targeting was synthesized (GenScript USA Inc. 860 Centennial Ave. Piscataway, NJ 08854). Guide RNA cassette for different target site was only different at the variable target sequences as described in the previous Examples. The variable target sequence in the guide RNA cassette always started with G, if G was not present in soy genome at the 1st position, a G substitution was applied.

### Example 9: Vector Cloning of Guide RNA Expression Cassettes, Cas9 Endonuclease Expression Cassettes and Donor DNA's for Introduction of Transgenic SSI Target Sites in a Soy Genomic Window

The soy U6-13.1 polymerase III promoter (SEQ ID NO: 113) was used to express guide RNAs that direct Cas9 nuclease to designated genomic target sites (Table 4 and 5). A soy codon optimized Cas9 endonuclease expression, a guide RNA expression, and a donor DNA (repair DNA) cassettes were cloned via a Gateway system to T-DNA backbone containing the transformation selection maker; the T-DNA was then electroporated to agrobacterial strain of AGL1 or LBA4404 thy-. The donor DNA contained FRT1/FRT6 (87, 12) sites flanking the AT-UBQ10 Promoter: PMI: UBQ14 terminator which upon gene integration by homologous recombination with the Cas9/gRNA system created the FRT1/FRT6 (87,12) target lines for SSI technology application. The CRISPR-Cas9 target site sequences from corresponding soy genomic site were placed outside of the homology arms to increase insertion efficiency.

The sequence of cassettes of guide RNA/Cas9 DNA targeting various soy genomic sites and donor DNA that were constructed for the introduction of transgenic SSI sites into Cas endonuclease target sites through homologous recombination are listed in Table 6 and Table 7. All the guide RNA/Cas9 constructs differed only in the 20 bp guide RNA variable targeting domain corresponding to the soy genomic target sites. All the donor DNA constructs differed in the two homologous regions such as arm1 (HDR1) and arm2 (HDR2) and CRISPR-Cas9 target sites. These guide RNA/Cas9 DNA constructs and donor DNAs were delivered into an elite soy genome 93Y21 by the Agrobacterium transformation procedure described in the previous Examples.

TABLE 6
Guide RNA/Cas9 and Donor DNAs used in soy transformation
for the Complex Trait Locus on Chr01.

| | Guide RNA | SEQ ID | | SEQ ID |
|---|---|---|---|---|
| Target site | Cassette | NOs: | Donor DNA cassette | NOs: |
| Chr01-TS2 | GM-U6-13.1 | 114 | 1TS2_HR1:GM-EF1A2 | 122 |
| | PRO:1TS2CR1 | | PRO:Intron::FRT1:PMI:UBQ14 | |
| | | | TERM:FRT6:1TS2_HR2 | |
| Chr01-T13 | GM-U6-13.1 | 115 | 1TS13_HR1:GM-EF1A2 | 123 |
| | PRO:1TS13CR1 | | PRO:Intron::FRT1:PMI:UBQ14 | |
| | | | TERM:FRT6:1TS13_HR2 | |
| Chr01-TS18 | GM-U6-13.1 | 116 | 1TS18_HR1:GM-EF1A2 | 124 |
| | PRO:1TS18CR1 | | PRO:Intron::FRT1:PMI:UBQ14 | |
| | | | TERM:FRT6:1TS18_HR2 | |
| Chr01-TS22 | GM-U6-13.1 | 117 | 1TS22_HR1:GM-EF1A2 | 125 |
| | PRO:1TS22CR1 | | PRO:Intron::FRT1:PMI:UBQ14 | |
| | | | TERM:FRT6:1TS22_HR2 | |
| Chr01-TS28 | GM-U6-13.1 | 118 | 1TS28_HR1:GM-EF1A2 | 126 |
| | PRO:1TS28CR1 | | PRO:Intron::FRT1:PMI:UBQ14 | |
| | | | TERM:FRT6:1TS28_HR2 | |
| Chr01-TS31 | GM-U6-13.1 | 119 | 1TS31_HR1:GM-EF1A2 | 127 |
| | PRO:1TS31CR1 | | PRO:Intron::FRT1:PMI:UBQ14 | |
| | | | TERM:FRT6:1TS31_HR2 | |
| Chr01-TS39 | GM-U6-13.1 | 120 | 1TS39_HR1:GM-EF1A2 | 128 |
| | PRO:1TS39CR1 | | PRO:Intron::FRT1:PMI:UBQ14 | |
| | | | TERM:FRT6:1TS39_HR2 | |
| Chr01-TS44 | GM-U6-13.1 | 121 | 1TS44_HR1:GM-EF1A2 | 129 |
| | PRO:1TS44CR1 | | PRO:Intron::FRT1:PMI:UBQ14 | |
| | | | TERM:FRT6:1TS44_HR2 | |

TABLE 7
Guide RNA/Cas9 and Donor DNAs used in soy transformation
for the Complex Trait Locus on Chr02.

| | Guide RNA | SEQ ID | | SEQ ID |
|---|---|---|---|---|
| Target site | Cassette | NOs: | Donor DNA cassette | NOs: |
| Chr02-TS2 | GM-U6-13.1 | 130 | 2TS2_HR1:GM-EF1A2 | 145 |
| | PRO:2TS2CR1 | | PRO:Intron::FRT1:PMI:UBQ14 | |
| | | | TERM:FRT6:2TS2_HR2 | |
| Chr02-TS4 | GM-U6-13.1 | 131 | 2TS4_HR1:GM-EF1A2 | 146 |
| | PRO:2TS4CR1 | | PRO:Intron::FRT1:PMI:UBQ14 | |
| | | | TERM:FRT6:2TS4_HR2 | |
| Chr02-TS5 | GM-U6-13.1 | 132 | 2TS5_HR1:GM-EF1A2 | 147 |
| | PRO:2TS5CR1 | | PRO:Intron::FRT1:PMI:UBQ14 | |
| | | | TERM:FRT6:2TS5_HR2 | |
| Chr02-TS7 | GM-U6-13.1 | 133 | 2TS7_HR1:GM-EF1A2 | 148 |
| | PRO:2TS7CR1 | | PRO:Intron::FRT1:PMI:UBQ14 | |
| | | | TERM:FRT6:2TS7_HR2 | |
| Chr02-TS8 | GM-U6-13.1 | 134 | 2TS8_HR1:GM-EF1A2 | 149 |
| | PRO:2TS8CR1 | | PRO:Intron::FRT1:PMI:UBQ14 | |
| | | | TERM:FRT6:2TS8_HR2 | |
| Chr02-TS11 | GM-U6-13.1 | 135 | 2TS11_HR1:GM-EF1A2 | 150 |
| | PRO:2TS11CR1 | | PRO:Intron::FRT1:PMI:UBQ14 | |
| | | | TERM:FRT6:2TS11_HR2 | |
| Chr02-TS13 | GM-U6-13.1 | 136 | 2TS13_HR1:GM-EF1A2 | 151 |
| | PRO2:TS13CR1 | | PRO:Intron::FRT1:PMI:UBQ14 | |
| | | | TERM:FRT6:2TS13_HR2 | |
| Chr02-TS20 | GM-U6-13.1 | 137 | 2TS20_HR1:GM-EF1A2 | 152 |
| | PRO:2TS20CR1 | | PRO:Intron::FRT1:PMI:UBQ14 | |
| | | | TERM:FRT6:2TS20_HR2 | |
| Chr02-TS26 | GM-U6-13.1 | 138 | 2TS26_HR1:GM-EF1A2 | 153 |
| | PRO:2TS26CR1 | | PRO:Intron::FRT1:PMI:UBQ14 | |
| | | | TERM:FRT6:2TS26_HR2 | |
| Chr02-TS29 | GM-U6-13.1 | 139 | 2TS29_HR1:GM-EF1A2 | 154 |
| | PRO:2TS29CR1 | | PRO:Intron::FRT1:PMI:UBQ14 | |
| | | | TERM:FRT6:2TS29_HR2 | |
| Chr02-TS37 | GM-U6-13.1 | 140 | 2TS37_HR1:GM-EF1A2 | 155 |
| | PRO:2TS37CR1 | | PRO:Intron::FRT1:PMI:UBQ14 | |
| | | | TERM:FRT6:2TS37_HR2 | |
| Chr02-TS41 | GM-U6-13.1 | 141 | 2TS41_HR1:GM-EF1A2 | 156 |
| | PRO:2TS41CR1 | | PRO:Intron::FRT1:PMI:UBQ14 | |
| | | | TERM:FRT6:2TS41_HR2 | |
| Chr02-TS45 | GM-U6-13.1 | 142 | 2TS45_HR1:GM-EF1A2 | 157 |
| | PRO:2TS45CR1 | | PRO:Intron::FRT1:PMI:UBQ14 | |
| | | | TERM:FRT6:2TS45_HR2 | |
| Chr02-TS51 | GM-U6-13.1 | 143 | 2TS51_HR1:GM-EF1A2 | 158 |
| | PRO:2TS51CR1 | | PRO:Intron::FRT1:PMI:UBQ14 | |
| | | | TERM:FRT6:2TS51_HR2 | |
| Chr02-TS57 | GM-U6-13.1 | 144 | 2TS57_HR1:GM-EF1A2 | 159 |
| | PRO:2TS57CR1 | | PRO:Intron::FRT1:PMI:UBQ14 | |
| | | | TERM:FRT6:2TS57_HR2 | |

### Example 10: Delivery of the Guide RNA/Cas9 Endonuclease System to Soy by Agrobacterium Transformation

CRISPR components were delivered to soy genome by a transformation procedure as described in WO2020 005933. The mature dry seed from soybean lines were surface-sterilized for sixteen hours using chlorine gas, produced by mixing 3.5 mL of 12 N HCl with 100 mL of commercial bleach (5.25% sodium hypochloride), as described by Di et al., (1996) Plant Cell Rep 15:746-750). Disinfected seeds were soaked in sterile distilled water at room temperature for sixteen hours (100 seeds in a 25×100 mm petri dish) and imbibed on semi-solid medium containing 5 g/L sucrose and 6 g/L agar at room temperature in the dark. After overnight incubation, the seeds were soaked in distilled water for an additional three to four hours at room temperature in the dark. Intact embryonic axes (EA) were isolated from cotyledons. Agrobacterium-mediated EA transformation was carried out as described herein and disclosed in U.S. patent application No. 20180216123. The compositions of various cultivation media used for soybean EA transformation and plant regeneration are summarized in WO2020 005933. A volume of 10 mL of Agrobacterium suspension (OD 0.50 at 600 nm) in infection medium containing 300 mM acetosyringone was added to the EA. The embryonic axes were co-cultivated with the Agrobacterium suspension containing the Agrobacterium vectors. The plates were sealed with parafilm (“Parafilm MTM” VWR Cat #52858), then sonicated (Sonicator-VWR model 50T) for thirty seconds. After sonication, about 90-500 embryonic axes were transferred to a single layer of autoclaved sterile filter paper (VWR #41 5/Catalog #28320-020). The plates were sealed with Micropore tape (Catalog #1530-0, 3M, St. Paul, MN)) and incubated under dim light (1-2 pE/m2/s), cool white fluorescent lamps for sixteen hours at 21° C. for three days. After co-cultivation, the base of each EA was embedded in shoot induction (SI) medium containing Spectinomycin 25 mg/L and 500 mg/L Cefotaxime. Shoot induction was carried out in a Percival Biological Incubator or growth room.

### Example 11: Detection of Target Site Mutation Mediated by the Guide RNA/Cas9 System in Transient Transformed Soy EA

The guide RNAs corresponding to the target sites at the two genomic chromosomes were first evaluated by a transient test. Transformation followed the same procedure as described in the previous examples but only up to shoot cluster formation on EAs. Shoot clusters from EAs after ten days infection, as described in the previous examples, were removed by scalpel. Six replicates each containing five EA shoot clusters for each guide/Cas9 vector (SEQ ID NO: 114-159) were obtained. Genomic DNA was extracted from shoot cluster samples using the Sbeadex™ plant DNA extraction kit (catalog #NAP41620, LGC, Biosearch Technologies, Middleton, WI 53562) following the plant extraction procedure from the vendor. Sequences across each CRISPR-Cas9 target site were amplified and the resulting amplicons were analyzed by next generation sequencing with reads not less than 1 million per sample. Genomic primers for amplification of each target site in the NGS are listed in Table 10. Mutation frequencies at each target site are listed in Table 8 and 9. Target site mutation frequency was measured by number of the total of mutation reads divided by the total number of reads. Since the majority of the shoot clusters was not transformed by the Agrobacterium in ten days, 0.4-5% mutation frequencies in transient test are considered a normal range. Twenty-one out of the twenty-three tested guides showed good mutation frequency and were suitable for insertion via stable transformation.

TABLE 8
Chromosome 1 Target site mutation.

| Target | Gen | Physical Location | Mutation |
|---|---|---|---|
| Site Name | Loc (cM) | 93Y21/w82.a2.v1 | Frequency (%) |
| TS2 | 13.28 | 1746967/1706570 | 0.8% |
| TS13 | 14.23 | 1806364/1763906 | 3.6% |
| TS18 | 14.63 | 1837616/1795168 | 1.9% |
| TS22 | 15.47 | 1903457/1861634 | 3.8% |
| TS28 | 17.27 | 2097350/2058947 | 1.4% |
| TS31 | 17.79 | 2172486/2134385 | 2.3% |
| TS39 | 18.91 | 2263922/2228268 | 1.2% |
| TS44 | 20.08 | 2356651/2321065 | 1.4% |

TABLE 9
Chromosome 2 target site mutation.

| Target | Gen | Physical Location | Mutation |
|---|---|---|---|
| Site Name | Loc (cM) | 93Y21/w82.a2.v1 | Frequency (%) |
| TS2 | 4 | 1194819/1165660 | 0.4% |
| TS4 | 4.11 | 1234962/1207215 | 0.1% |
| TS5 | 4.12 | 1237306/1209559 | 0.6% |
| TS7 | 5.04 | 1370919/1344278 | 2.2% |
| TS8 | 5.05 | 1372628/UNKNOWN | 2.2% |
| TS11 | 5.46 | 1438275/1419818 | 0.60% |
| TS13 | 7.13 | 1598311/1587139 | 0.9% |
| TS20 | 7.64 | 1678616/1667338 | 0.1% |
| TS26 | 8.04 | 1743109/1731691 | 0.5% |
| TS29 | 8.15 | 1761068/1749747 | 0.8% |
| TS37 | 8.4 | 1801537/1793127 | 2.0% |
| TS41 | 8.42 | 1803499/1795089 | 0.8% |
| TS45 | 9.15 | 1990238/1985597 | 2.2% |
| TS51 | 9.23 | 2002443/1997808 | 0.4% |
| TS57 | 9.74 | 2081706/2077352 | 0.9% |

### Example 12: Detection of Transgenic SSI Target Sites (SSILP) at the CRISPR Sites in the Soy Complex Trait Loci

A subset of target sites at the two chromosomal regions were selected to introduce the SSI landing pad (SSILP) via homology directed DNA repair to establish SSI target sites, the sites then can be used to integrate desired trait genes via FLP recombinase mediated cassette exchange (Gao, et al., 2020). Transformation followed the procedure as described in the previous examples, after shoot formation, and rooting, TO plants were produced and grew in soil in flats in control environment. Approximately, 500 to 750 TO plants were produced when targeting each Cas9 site. Leaf punch was collected from each TO plant, DNA was extracted using the same procedures as described in Example 11. Junction PCR with the Phusion Flash High Fidelity PCR Master Mix™ (#F-548L Thermo Scientific) were used to detect SSILP insertion in the TO plants. Plants positive for both 5′ and 3′ junctions (hereafter referred to as 2×HDR) were selected to be transferred to pots and grow the maturity. Insertion frequency (Table 10) at each site was measured by number of 2×HDR TO plant divided by total number TO plants analyzed. At each junction, one primer is on the genomic sequence outside of the sequence homology to the HDR arm in the donor, and the other sequence is on the donor, the primer sequences are listed in Table 11. Next, the 2×HDR TO plants were transferred to pots and are selfed to produce T1 seeds.

T1 seeds were planted in flats and leaf punches were collected, DNA was processed using same method as for TO plants. Junction PCRs were performed to confirm SSILP insertion at each target site. Additionally, qPCRs were also performed on the T1 samples. The qPCRs are to check if T-DNA present in the T1 plants, Cas9, guide, selection marker, Ds-RED on T-DNA are checked for present or absent. Soy codon optimized phosphomannose isomerase (PMI) was included in each SSILP and copy number of PMI was used to identify homozygous or hemizygous insertion at the CRISPR site. The T1 plants with SSILP insertion at target site and free of T-DNA based on qPCR assays were further analyzed using Southern by Sequencing (SbS). Plants with perfect SSILP insertion at the CRISPR target site and without any T-DNA insertion in any part of the genome are referred to as SbS green plants.

TABLE 10
CRISPR-Cas9 mediated insertion of SSILP
selected sites in soy complex trait loci

| | Target Site | | GenPos | 2 × HDR |
|---|---|---|---|---|
| Chromosome | Name | PhysicalPos | (cM) | Frequency |
| 1 | Chr01-TS2 | 1746967 | 14.04 | 0.6% |
| 1 | Chr01-TS18 | 1837616 | 15.17 | 6.3% |
| 1 | Chr01-TS31 | 2172486 | 18.27 | 1.0% |
| 2 | Chr02-TS2 | 1194819 | 4.0 | 3.6% |
| 2 | Chr02-TS5 | 1237306 | 4.12 | 2.6% |
| 2 | Chr02-TS8 | 1372628 | 5.05 | 2.2% |
| 2 | Chr02-TS11 | 1438275 | 5.46 | 0.8% |
| 2 | Chr02-TS13 | 1598311 | 7.13 | 5.7% |
| 2 | Chr02-TS26 | 1743109 | 8.04 | 5.1% |
| 2 | Chr02-TS29 | 1761068 | 8.15 | 1.8% |
| 2 | Chr02-TS37 | 1801537 | 8.4 | 1.2% |
| 2 | Chr02-TS45 | 1990238 | 9.15 | 1.4% |
| 2 | Chr02-TS57 | 2081706 | 9.74 | 1.3% |

The PMI sequence in the donor of the following loci Chr01-TS2, Chr02-TS18, Chr01-tS31, and Chr01-TS37 sites were different than earlier provided, this PMI sequence was codon optimized using soybean preferred codons. This PMI sequence was used in the stable transformation and the sequences are provided in SEQ ID NO:208, 209, 210, and 211.

TABLE 11
Primer sequences to amplify the junctions
of SSILP to each CRISPR/Cas9 target site

| | Target Site | Primer | 5′ Junction | 3′ Junction |
|---|---|---|---|---|
| Chromosome | Name | orientation | SEQ ID NO: | SEQ ID NO: |
| 1 | Chr01-TS2 | forward | 212 | 238 |
| 1 | Chr01-TS2 | reverse | 213 | 239 |
| 1 | Chr01-TS18 | forward | 214 | 240 |
| 1 | Chr01-TS18 | reverse | 215 | 241 |
| 1 | Chr01-TS31 | forward | 216 | 242 |
| 1 | Chr01-TS31 | reverse | 217 | 243 |
| 2 | Chr02-TS2 | forward | 218 | 244 |
| 2 | Chr02-TS2 | reverse | 219 | 245 |
| 2 | Chr02-TS5 | forward | 220 | 246 |
| 2 | Chr02-TS5 | reverse | 221 | 247 |
| 2 | Chr02-TS8 | forward | 222 | 248 |
| 2 | Chr02-TS8 | reverse | 223 | 249 |
| 2 | Chr02-TS11 | forward | 224 | 250 |
| 2 | Chr02-TS11 | reverse | 225 | 251 |
| 2 | Chr02-TS13 | forward | 226 | 252 |
| 2 | Chr02-TS13 | reverse | 227 | 253 |
| 2 | Chr02-TS26 | forward | 228 | 254 |
| 2 | Chr02-TS26 | reverse | 229 | 255 |
| 2 | Chr02-TS29 | forward | 230 | 256 |
| 2 | Chr02-TS29 | reverse | 231 | 257 |
| 2 | Chr02-TS37 | forward | 232 | 258 |
| 2 | Chr02-TS37 | reverse | 233 | 259 |
| 2 | Chr02-TS45 | forward | 234 | 260 |
| 2 | Chr02-TS45 | reverse | 235 | 261 |
| 2 | Chr02-TS57 | forward | 236 | 262 |
| 2 | Chr02-TS57 | reverse | 237 | 263 |

### Example 13: Detection of Gene Expression at the SSI Target Sites

For useful transgenic trait development, genomic sites must be able to support transgene expression. To assess the effect of insertion site on transgene expression, protein expression levels of the phosphomannose isomerase (PMI, or PMI (SO1)) gene in SSILPs were measured using ELISA in T2 plant leaves. Sixteen homozygous seeds from each site were randomly planted in flats, and leaf samples were collected at 4 leave stage. Table 12 showed PMI (SO1) expression at the 3 tested sites, the three sites all support gene expression. Additional expression for subsequent generations of plants (for example T2, T3, T4) is completed and confirms that the protein expression of PMI at the soy CTLs remains robust in progeny plants.

TABLE 12
Protein expression of the PMI gene at
three tested targets in the soy CTL

| Target Site Name | PhysicalPos | GenPos (cM) | PMI (ppm) |
|---|---|---|---|
| Chr02-TS11 | 1438275 | 5.46 | 8783 ± 859 |
| Chr02-TS26 | 1743109 | 8.04 | 5473 ± 317 |
| Chr02-TS45 | 1990238 | 9.15 | 7016 ± 593 |

### Example 14: Cas12fl Mediated Insertion within Soybean Complex Trait Loci

Cas endonuclease from Cas endonuclease Syntrophomonas palmitatica Cas12fl (SpaCas12fl) can also be used to making double stranded breaks in genome and facilitate genome editing. It recognized a NTTC at 5′ PAM. Syntrophomonas palmitatica Cas endonuclease target sites (71 sites) within the genomic window in the second soy genomic window spans from Chr02:1117000 to Chr02:2129999 on chromosome 2 were identified as sites which are at least 2-2.5 kb away from any known gene's transcription start site and at least 500 bp preferably 1 kb or 2 kb away from repetitive sequences. Table 13 shows the physical and genetic map position of the sites (Ganal, M. et al (2011) PloS one, DOI: 10.1371).

TABLE 13
CRISPR-Cas12fl mediated insertion sites in soy complex trait loci.

| | | PAM | | PAM | PAM |
|---|---|---|---|---|---|
| Name of Cas | SpCas12f1 | Physical | Genetic | Physical | Physical |
| endonuclease | target site | position | Position | position | position |
| target site | SEQ ID No | (93Y21) | (cM) | (93Y21) | (w82.a2.v1) |
| Chr02-TS58 | 264 | 1193220 | 3.99 | 1193220 | 1165471 |
| Chr02-TS59 | 265 | 1204819 | 4.03 | 1204819 | 1177069 |
| Chr02-TS60 | 266 | 1206671 | 4.03 | 1206671 | 1178921 |
| Chr02-TS61 | 267 | 1219261 | 4.07 | 1219261 | 1191512 |
| Chr02-TS62 | 268 | 1221607 | 4.07 | 1221607 | 1193860 |
| Chr02-TS63 | 269 | 1232454 | 4.10 | 1232454 | 1204707 |
| Chr02-TS64 | 270 | 1233097 | 4.11 | 1233097 | 1205350 |
| Chr02-TS65 | 271 | 1237861 | 4.12 | 1237861 | 1210114 |
| Chr02-TS66 | 272 | 1295627 | 4.47 | 1295627 | 1268844 |
| Chr02-TS67 | 273 | 1295716 | 4.47 | 1295716 | 1268933 |
| Chr02-TS68 | 274 | 1366247 | 5.00 | 1366247 | 1339417 |
| Chr02-TS69 | 275 | 1368655 | 5.02 | 1368655 | 1342014 |
| Chr02-TS70 | 276 | 1370897 | 5.04 | 1370897 | 1344256 |
| Chr02-TS71 | 277 | 1372150 | 5.05 | 1372150 | not in this line |
| Chr02-TS72 | 278 | 1372747 | 5.05 | 1372747 | not in this line |
| Chr02-TS73 | 279 | 1380628 | 5.11 | 1380628 | 1362080 |
| Chr02-TS74 | 280 | 1383098 | 5.12 | 1383098 | 1364550 |
| Chr02-TS75 | 281 | 1385925 | 5.14 | 1385925 | 1367377 |
| Chr02-TS76 | 282 | 1397753 | 5.22 | 1397753 | 1379305 |
| Chr02-TS77 | 283 | 1400187 | 5.23 | 1400187 | 1381735 |
| Chr02-TS78 | 284 | 1438038 | 5.46 | 1438038 | 1419581 |
| Chr02-TS79 | 285 | 1438271 | 5.46 | 1438271 | 1419814 |
| Chr02-TS80 | 286 | 1476509 | 5.64 | 1476509 | 1458054 |
| Chr02-TS81 | 287 | 1490466 | 5.89 | 1490466 | 1471996 |
| Chr02-TS82 | 288 | 1491659 | 5.91 | 1491659 | 1473189 |
| Chr02-TS83 | 289 | 1491816 | 5.92 | 1491816 | 1473346 |
| Chr02-TS84 | 290 | 1547141 | 6.63 | 1547141 | 1535986 |
| Chr02-TS85 | 291 | 1584251 | 7.02 | 1584251 | 1573083 |
| Chr02-TS86 | 292 | 1598304 | 7.13 | 1598304 | 1587132 |
| Chr02-TS87 | 293 | 1641558 | 7.40 | 1641558 | 1630368 |
| Chr02-TS88 | 294 | 1644889 | 7.43 | 1644889 | 1633699 |
| Chr02-TS89 | 295 | 1649930 | 7.46 | 1649930 | 1638742 |
| Chr02-TS90 | 296 | 1674988 | 7.61 | 1674988 | 1663718 |
| Chr02-TS91 | 297 | 1714878 | 7.86 | 1714878 | 1703577 |
| Chr02-TS92 | 298 | 1743161 | 8.04 | 1743161 | 1731743 |
| Chr02-TS93 | 299 | 1757257 | 8.13 | 1757257 | 1745872 |
| Chr02-TS94 | 300 | 1757370 | 8.13 | 1757370 | 1745985 |
| Chr02-TS95 | 301 | 1761682 | 8.16 | 1761682 | 1750361 |
| Chr02-TS96 | 302 | 1762192 | 8.16 | 1762192 | 1750871 |
| Chr02-TS97 | 303 | 1763444 | 8.17 | 1763444 | 1752123 |
| Chr02-TS98 | 304 | 1764760 | 8.17 | 1764760 | 1753439 |
| Chr02-TS99 | 305 | 1767607 | 8.19 | 1767607 | 1756272 |
| Chr02-TS100 | 306 | 1767630 | 8.19 | 1767630 | 1756295 |
| Chr02-TS101 | 307 | 1769141 | 8.20 | 1769141 | 1757806 |
| Chr02-TS102 | 308 | 1771353 | 8.22 | 1771353 | 1760018 |
| Chr02-TS103 | 309 | 1787281 | 8.32 | 1787281 | 1775947 |
| Chr02-TS104 | 310 | 1794716 | 8.36 | 1794716 | 1783477 |
| Chr02-TS105 | 311 | 1802127 | 8.41 | 1802127 | 1793717 |
| Chr02-TS106 | 312 | 1859478 | 8.74 | 1859478 | 1851138 |
| Chr02-TS107 | 313 | 1861219 | 8.75 | 1861219 | 1852879 |
| Chr02-TS108 | 314 | 1958394 | 9.00 | 1958394 | 1950123 |
| Chr02-TS109 | 315 | 1988353 | 9.14 | 1988353 | not in this line |
| Chr02-TS110 | 316 | 1996037 | 9.19 | 1996037 | 1991404 |
| Chr02-TS111 | 317 | 1996328 | 9.19 | 1996328 | 1991693 |
| Chr02-TS112 | 318 | 1996502 | 9.19 | 1996502 | 1991867 |
| Chr02-TS113 | 319 | 2002035 | 9.23 | 2002035 | 1997400 |
| Chr02-TS114 | 320 | 2002610 | 9.23 | 2002610 | 1997975 |
| Chr02-TS115 | 321 | 2002615 | 9.23 | 2002615 | 1997980 |
| Chr02-TS116 | 322 | 2002631 | 9.23 | 2002631 | 1997996 |
| Chr02-TS117 | 323 | 2002642 | 9.23 | 2002642 | 1998007 |
| Chr02-TS118 | 324 | 2010999 | 9.29 | 2010999 | 2006440 |
| Chr02-TS119 | 325 | 2022615 | 9.36 | 2022615 | 2018074 |
| Chr02-TS120 | 326 | 2023049 | 9.36 | 2023049 | 2018508 |
| Chr02-TS121 | 327 | 2029955 | 9.41 | 2029955 | 2025414 |
| Chr02-TS122 | 328 | 2038836 | 9.47 | 2038836 | 2034358 |
| Chr02-TS123 | 329 | 2038925 | 9.47 | 2038925 | 2034447 |
| Chr02-TS124 | 330 | 2043048 | 9.49 | 2043048 | 2038570 |
| Chr02-TS125 | 331 | 2081497 | 9.74 | 2081497 | 2077143 |
| Chr02-TS126 | 332 | 2081573 | 9.74 | 2081573 | 2077219 |
| Chr02-TS127 | 333 | 2083970 | 9.76 | 2083970 | 2079616 |
| Chr02-TS128 | 334 | 2084631 | 9.76 | 2084631 | 2080277 |

While the present disclosure may be susceptible to various modifications and alternative forms, specific embodiments have been described by way of example in detail herein. However, it should be understood that the present disclosure is not intended to be limited to the particular forms disclosed. Rather, the present disclosure is to cover all modifications, equivalents, and alternatives falling within the scope of the present disclosure as defined by the following appended claims and their legal equivalents.

1. A soybean genomic integration site, said integration site comprising the following characteristics:
a. greater than 5 Kb in size;
b. low genetic diversity, wherein the low genetic diversity comprises a haplotype with greater than 80% frequency;
c. high expected recombination frequencies, wherein the recombination frequencies are greater than 0.7 cM/1 Mb; and
d. close proximity to a telomere, wherein the close proximity is less than 20 cM or less than 4.7 Mb from the end of a chromosome.

2. The soybean genomic integration site of claim 1, wherein the genomic integration site comprises SEQ ID NO:206 or SEQ ID NO:207.

3. The soybean genomic integration site of claim 1, wherein the genomic integration site comprises SEQ ID NO:1-109 or SEQ ID NO:264-334.

4. The soybean genomic integration site of claim 1, wherein the Genetic size of the low-diversity genomic region is less than 10 cM.

5. The soybean genomic integration site of claim 1, wherein the Physical size of the low-diversity genomic region is less than 2.6 Mb.

6. The soybean genomic integration site of claim 1, wherein the integration site comprises euchromatin.

7. The soybean genomic integration site of claim 1, wherein the integration site is greater than 10 cM or 1.05 Mb in distance from heterochromatin.

8. The soybean genomic integration site of claim 1, wherein the range of the Haplotype frequency is from 80 to 100%.

9. The soybean genomic integration site of claim 1, wherein the integration site is greater than 5 cM from a QTL.

10. The soybean genomic integration site of claim 1, wherein the integration site does not occur in a region that contains Structural Variation that inhibits genomic recombination.

11. The soybean genomic integration site of claim 1, wherein the integration site comprises a gene expression cassette.

12. The soybean genomic integration site of claim 11, wherein the gene expression cassette comprises an insecticidal resistance gene, herbicide tolerance gene, nitrogen use efficiency gene, water use efficiency gene, nutritional quality gene, DNA binding gene, and/or selectable marker gene.

13. The soybean genomic integration site of claim 1, wherein said integration site comprises at least one target site.

14. The soybean genomic integration site of claim 13, wherein said target site is cleaved by a site specific nuclease.

15. The soybean genomic integration site of claim 14, wherein said site specific nuclease is selected from the group consisting of a zinc finger nuclease, a CRISPR nuclease, a TALEN, a homing endonuclease or a meganuclease.

16. The soybean genomic integration site of claim 1, wherein said integration site sequences are modified during insertion of a donor DNA into said integration site sequence.

17. A method of making a transgenic soybean plant cell comprising a donor DNA targeted to a genomic integration site, the method comprising:
a. selecting a target site within the genomic integration site;
b. introducing a site specific nuclease into a plant cell, wherein the site specific nuclease cleaves said target site;
c. introducing the donor DNA into the plant cell;
d. targeting the donor DNA into said target site of the genomic integration site, wherein the cleavage of said target site facilitates integration of the donor DNA into said target site; and
e. selecting transgenic plant cells comprising the donor DNA targeted to said target site of the genomic integration site.

18. The method of making a transgenic soybean plant cell of claim 17, wherein said donor DNA comprises a gene expression cassette.

19. The method of making a transgenic soybean plant cell of claim 18, wherein the gene expression cassette comprises an insecticidal resistance gene, herbicide tolerance gene, nitrogen use efficiency gene, water use efficiency gene, nutritional quality gene, DNA binding gene, and/or selectable marker gene.

20. The method of making a transgenic soybean plant cell of claim 17, wherein said site specific nuclease is selected from the group consisting of a zinc finger nuclease, a CRISPR nuclease, a TALEN, a homing endonuclease or a meganuclease.

21. A method of identifying a soybean genomic integration site for site specific nuclease meditated integration of a donor polynucleotide, comprising the steps of:
a. obtaining a soybean genomic DNA sample from at least one soybean variety;
b. genotyping the soybean genomic DNA sample;
c. identifying haplotypes of the soybean genomic DNA sample;
d. assessing the genetic diversity of the haplotypes to identify haplotypes with low genetic diversity;
e. selecting haplotypes with low genetic diversity that comprises the following characteristics:
1. greater than 1 cM in size;
2. low genetic diversity, wherein the low genetic diversity comprises a haplotype with greater than 80% frequency;
3. high expected recombination frequencies, wherein the recombination frequencies are greater than 0.7 cM/1 Mb; and,
4. close proximity to a telomere, wherein the close proximity is less than 20 cM or less than 4.7 Mb from the end of a chromosome;
wherein the soybean genomic integration site is targeted with a site-specific nuclease to integrate a donor polynucleotide within the soybean genomic integration site.
